(19) United States
(12) Patent Application Publication
(10) Pub. No.: US 2010/0303723 A1
(43) Pub. Date: Dec. 2, 2010

(54) DRUG DELIVERY SYSTEMS USING FC FRAGMENTS

(75) Inventors: Omid C. Farokhzad, Chestnut Hill, MA (US); Frank Alexis, Brighton, MA (US); Timothy T. Kuo, Chestnut Hill, MA (US); Eric Pridgen, Boston, MA (US); Aleksandar Filip Radovic-Moreno, State College, PA (US); Robert S. Langer, Newton, MA (US)

Correspondence Address: Pabst Patent Group LLP, 1545 PEACHTREE STREET NE, SUITE 320, ATLANTA, GA, 30309, US

(73) Assignees: MASSACHUSETTS INSTITUTE OF TECHNOLOGY, Cambridge, MA (US); THE BRIGHAM AND WOMEN'S HOSPITAL, INC., Boston, MA (US)

(21) Appl. No.: 12/515,465

(22) PCT Filed: Nov. 20, 2007
(86) PCT No.: PCT/US07/85318

§ 371 Date: May 5, 2010

(51) Int. Cl. A61K 9/127; A61K 9/14; A61K 39/395; A61K 49/00; A61K 38/21; C07K 16/00; C12N 9/96; C07K 14/555; A61P 9/00

(52) U.S. Cl. 424/9.1; 424/491; 424/178.1; 424/85.4; 424/130.1; 424/141.1; 424/133.1; 424/450; 530/391.7; 435/188; 530/351; 977/774

(57) ABSTRACT

The present invention provides drug delivery systems comprising FcRn binding partners (e.g., FcRn binding partner, Fc fragment) associated with a particle or an agent to be delivered. Inventive drug delivery systems allow for binding to the FcRn receptor and transcytosis into and/or through a cell or cell layer. Inventive systems are useful for delivering therapeutic agents across the endothelium of blood vessels or the epithelium of an organ.

### RELATED APPLICATIONS

This application claims priority under 35 U.S.C. §119(e) to U.S. provisional patent application, U.S. Ser. No. 60/860,043, filed Nov. 20, 2006 (“the '043 application”). The entire contents of the '043 application are incorporated herein by reference.

### GOVERNMENT SUPPORT

The United States Government has provided grant support utilized in the development of the present invention. In particular, National Institute of Health (contract number CA119349) has supported development of this invention. The United States Government has certain rights in the invention.

### BACKGROUND OF THE INVENTION

Targeted delivery for diagnostic and therapeutic applications has until recently largely been limited to receptor ligands such as peptides, nucleic acids, and antibodies fragments to deliver agents intracellularly and/or to specific targets. Antibodies are the most widely used type of targeting agent today. The large size of antibody molecules can make it difficult to transport targeting systems across cellular membranes. In some instances, large targeting systems can lead to slow elimination from the blood circulation, which can ultimately lead to myelotoxicity. In addition, in vivo use of antibody-based targeting systems is expensive and can lead to immunogenicity after repeated injections of such formulations. Antibody fragments which are smaller than whole antibodies have successfully been made but are still, in many instances, too large. Fragments can reach extracellular spaces more easily than whole antibodies. However, there is no drug delivery system that can specifically and dynamically be used to transport across cell layers without relying on additives, chemical stress, mechanical stress, or electrical stress.

### SUMMARY OF THE INVENTION

This invention is generally in the field of drug delivery systems. The system includes the use of FcRn binding partners as targeting moieties conjugated to a biodegradable polymer and formation of functionalized particles that can be transported across a cell or cellular layer. Here we disclose antibodies or fragments thereof used as targeting moieties and receptor mediated transport for a controlled drug release system.

The FcRn receptor molecule is well characterized. The FcRn receptor binds IgG (but not other immunoglobulin classes such as IgA, IgD, IgM, and/or IgE) at acidic pH and not at basic pH. FcRn transports IgG across epithelial cells either in the direction of apical to basolateral surface or in the direction of basolateral to apical surface. As will be recognized by those of ordinary skill in the art, FcRn receptors and/or characteristic portions thereof can be isolated by cloning or by affinity purification using, for example, monoclonal antibodies. Such isolated FcRn receptors then can be used to identify and/or isolate FcRn binding partners, as described below. FcRn binding partners include whole IgG, the Fc fragment of IgG, and/or other fragments of IgG that include the complete binding region for the FcRn receptor. The region of the Fc portion of IgG that binds to the FcRn receptor has been described based upon X-ray crystallography (Burmeister, et al., 1994, Nature, 372:379; incorporated herein by reference). The major contact area of Fc with the FcRn receptor is near the junction of the CH2 and CH3 domains. Potential contacts are residues 248, 250-257, 272, 285, 288, 290-291, 308-311, and/or 314 in CH2 and 385-387, 428, and/or 433-436 in CH3. (These sites are distinct from those identified by subclass comparison or by site-directed mutagenesis as important for Fc binding to leukocyte FcγRI and FcγRII.). The foregoing Fc-FcRn contacts are all within a single Ig heavy chain. It has been noted previously that two FcRn receptors can bind a single Fc molecule. The crystallographic data suggest that in such a complex, each FcRn molecule binds a single polypeptide of the Fc homodimer. The Fc region of IgG can be modified to yield modified Fc fragments or portions thereof that will be bound by the FcRn receptor. Such modifications include modifications remote from the FcRn contact sites as well as modifications within the contact sites that preserve or even enhance binding. In some embodiments, other binding partners can be identified and isolated. Antibodies or portions thereof specific for the FcRn receptor and capable of being transported by FcRn once bound can be identified and isolated using well established molecular biology-based techniques. Likewise, libraries of peptides, polynucleotides, or small molecules can be screened and molecules that are bound and transported by FcRn receptors can be isolated using conventional techniques. It is not intended that the invention be limited by the selection of any particular FcRn binding partner. Where the binding partner is IgG or an FcRn binding portion thereof, the IgG or portion thereof may be prepared according to conventional procedures. The present invention relates to FcRn binding partners for the targeted delivery of vaccines, antigens, drugs, therapeutics, microparticles, nanoparticles, picoparticles, etc. to and/or across epithelial and/or endothelial barriers. Previous attempts to use targeted drug delivery have been limited by an inability to effectively cross epithelial cell layers. By using FcRn targeted nanoparticles, it may be possible to enhance delivery across cells, layers of cells, and/or tissues, resulting in improved drug distribution and targeting

The invention is useful whenever it is desirable to achieve systemic, oral, or local delivery of a diagnostic agent (e.g., fluorescent or radiopague compound) or therapeutic agent (e.g., a drug or chemical), delivery vehicle, protein, polynucleotide, and/or combinations thereof across an epithelial barrier into the systemic circulation, or from the systemic circulation across the endothelial barrier. The invention may be used to administer a therapeutic agent to elicit a beneficial effect. The FcRn binding partner conjugates are designed to deliver a wide variety of therapeutics including RNA and DNA nucleotides (as used, for example, in gene therapy), peptides, carbohydrates, and/or small molecules or chemical compounds. These therapeutics include, but are not limited to, anticancer and chemotherapeutic drugs, e.g., doxorubicin; anti-inflammatory drugs, e.g., steroids; drugs for the treatment of cardiovascular disease, e.g., cholinesterase inhibitors; drugs for the treatment of disorders related to viral infection, e.g., hepatic cirrhosis resulting from hepatitis infection; drugs for the treatment of weight disorders, e.g., amphetamines; antibacterial agents, antifungal agents, cytokines, fertility drugs, antibiotics, hormones, steroids, etc. In certain embodiments, the drug delivery system is used to deliver a protein therapeutic or prophylatic agent.

In one aspect, the invention provides an FcRn binding partner (e.g., Fc fragment) conjugated to a drug delivery system (e.g., polymeric particles such as nanoparticles or microparticles; liposomes; dendrimers; DNA- and/or RNA-containing particles; genetically engineered viral particles; inorganic particles; protein particles, etc.) that is able to transfer the polymeric particles with their payloads across endothelial and/or epithelial cell layers via a transcytosis mechanism. This invention includes pharmaceutical compositions that include these particles, methods of preparing the inventive drug delivery system, and methods for their use. Inventive drug delivery systems include a therapeutic, diagnostic, prognostic, or prophylactic agent(s) to be delivered. In certain embodiments, an FcRn binding partner (e.g., Fc fragment, such as an IgG Fc fragment) is linked to the controlled release polymer system (i.e., particles); binds selectively to the FcRn receptor on a cell, such as an endothelial and/or cell surface receptor; and thereby causes the delivery of the particles across the cell or cell layer.

In some embodiments, after a drug delivery system comprising an FcRn binding partner (e.g., Fc fragment) is delivered to the interior of a cell, the FcRn binding partner (e.g., Fc fragment) can target the drug delivery system to immune system components through FcRn receptor on cells of the immune system (e.g. macrophages). In some cases, it may be desirable to target a drug delivery system to immune system components (e.g., for vaccination). In some embodiments, it may not be desirable to target a drug delivery system to immune system components.

In some embodiments, drug delivery systems in accordance with the present invention comprise FcRn binding partners that may be shed once the drug delivery system has reached its target. For example, an FcRn binding partner may be shed from the drug delivery system upon reaching the interior of a cell. In some embodiments, an FcRn binding partner may be shed from the drug delivery system upon reaching the cellular surface. In some embodiments, an FcRn binding partner may not be shed from the drug delivery system at any point during or after drug delivery. In certain embodiments, an FcRn binding partner is shed once the drug delivery system reaches the bloodstream. In certain embodiments, an FcRn binding partner is shed once the drug delivery system has been transcytosed. This shedding of the FcRn binding partner may be accomplished in various ways including associating the FcRn binding partner with the drug delivery system using a cleavable linker (e.g., a peptide linker, disulfide linker, ester-containing linker). The peptide linker, for example, may be cleaved by protease found on the cell surface, in the extracellular matrix, or in the bloodstream.

Drug delivery systems comprising an FcRn binding partner are useful in preparing pharmaceutical composition. Such composition can be administered orally, parenterally, inhalationally, intravascularly, systemically, and/or locally. In certain particular embodiments, controlled release polymer systems associated with Fc fragments are delivered to the endothelium of the cardiovasculature (e.g., the coronary arteries) using a balloon catheter or another delivery device. Local delivery of therapeutic agents at a site of arterial injury rather than by systemic administration has been discussed previously (Labhasetwar et al., 1997, Adv. Drug Del. Rev., 24:63; incorporated herein by reference). Experimental studies in animal models of restenosis have been used to investigate local delivery of therapeutics for the prevention of restenosis (Lambert et al., 1994, Circulation, 90:1003; Lambert et al., 1994, Coron. Artery Dis., 5:163; Garcia et al., 1990, Surg. Gynecol. Obstet., 171:201; Edelman et al., 1990, Proc. Nat. Acad. Sci., USA, 87:3773; Edelman et al., 1993, Proc. Nat. Acad. Sci., USA, 90:1513; Edelman and Karnovsky, 1994, Circulation, 89:770; Nathan et al., 1995, Proc. Nat. Acad. Sci., USA, 92:8130; Okada et al. 1989, Neurosurgery 25:892; Villa et al., 1994, J. Clin. Invest., 93:1243; and Villa et al., 1995, Circ. Res., 76:505; all of which are incorporated herein by reference). Adventitial drug implants (Edelman et al., 1990, supra; Villa et al., 1994, supra; Simons et al., 1992, Nature, 359:67; Simons et al., 1994, J. Clin. Invest., 93:2351; all of which are incorporated herein by reference), stents (Lincoff et al., 1994, J. Am. Coll. Cardiol., 23:18A; and Jeong et al., 1994, Circulation, 92:I37; both of which are incorporated herein by reference), and catheter-based delivery systems (Steg et al., 1994, Circulation 90:1648; and Fernandez et al., 1994, Circulation 89:1518; both of which are incorporated herein by reference) have been disclosed. Lanza et al. (2002, Circulation 106:2842; incorporated herein by reference) teach targeted paramagnetic nanoparticles containing paclitaxel for the prevention of restenosis after angioplasty. The challenge has always been to develop particles that can specifically bind to and get taken across the endothelial barrier. The current invention provides a drug delivery system that is able to cross the endothelial barrier more effectively than previous systems.

The present invention generally relates to the preparation and use of a drug delivery system with an FcRn binding partner to deliver agents across a cell layer. In certain embodiments, the drug delivery system is a conjugate that includes a therapeutic agent conjugated to an FcRn binding partner (e.g., an IgG Fc fragment). In certain embodiments, a diagnostic agent such as a contrast agent is conjugated to an FcRn binding partner (e.g., an IgG Fc fragment). In certain embodiments, the agent to be delivered is a protein or peptide, and the conjugate is a fusion protein comprising the agent to be delivered and an IgG Fc fragment. In certain embodiments, the drug delivery system is a particle that is physically associated with a therapeutic agent and an FcRn binding partner (e.g., an IgG Fc fragment). Such systems are useful for preparing pharmaceutical compositions, for example, for oral delivery, inhalational delivery, parenteral delivery, intravascular delivery, systemic delivery, and/or the local delivery of agents.

In some embodiments, the invention relates to methods and compositions for the delivery of therapeutic agents conjugated to an FcRn binding partner to epithelial or endothelial cells (e.g., intestinal epithelium, mucosal epithelium, epithelium of the lung, epithleium of the liver, endothelium of the skin and vascular tissues, etc.). Conjugates of the invention (e.g., particles conjugated to an IgG Fc fragment; a small molecule or peptide conjugated to an IgG Fc fragment; etc.) can be used to deliver one or more agents across an endothelial and/or epithelial cell layer by contacting the FcRn receptor of the cell with the particle or conjugate. An effective amount of conjugate is delivered to achieve the desired result (e.g., treat a disease, prevent a disease, image a tissue, diagnosis a pathological condition, etc.). In certain embodiments, conjugates are delivered using a device for local, systemic, inhalational, intravascular, and/or oral drug delivery. In certain embodiments, conjugates are delivered to the endothelium of the cardiovasculature (e.g., the coronary arteries) using a balloon catheter. The invention takes advantage of receptor-mediated transport to deliver a payload across the endothelial and/or epithelial cell layer of the targeted tissue.

In some embodiments, the invention provides methods of conjugating the FcRn binding partner (e.g., Fc fragment) to a controlled release drug delivery system. For example, the invention provides methods of conjugating an FcRn binding partner (e.g., Fc fragment) to a drug delivery polymeric particle. The invention provides methods for conjugating an FcRn binding partner to a small molecule, protein, peptide, polynucleotide, and/or other agent to be delivered across a cell layer. Any isotypes of IgG and IgG Fc fragments may be used. The Fc fragment may be modified. In certain embodiments, an Fc fragment is at least 50%, at least 60%, at least 70%, at least 80%, at least 90%, at least 95%, at least 98%, or at least 99% homologous to a human IgG Fc fragment. The Fc fragment can be attached to a particle using any means known in the art. In certain specific embodiments, the attachment is a covalent attachment (e.g., an amide, an ester, disulfide, or other “click” chemistry), which may optionally comprise a linker (e.g., a peptide linker). In certain embodiments, an activated ester on the particle or agent to be delivered is allowed to react with a nucleophile such as a primary amine (e.g., terminal amine, lysine) of the Fc fragment. In some embodiments, the attachment is non-covalent based on affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

In some embodiments, the invention provides kits for the use of the inventive drug delivery systems. Kits may include one or more doses of a drug delivery system for administration to a subject. In certain embodiments, a kit includes a device for delivering the drug delivery system including a syringe, a needle, a catheter, tubing, solutions, buffers, etc. A kit typically includes instructions for administering drug delivery systems. The convenient packaging of a kit allows for the easy use of the drug delivery system or pharmaceutical compositions thereof.

### BRIEF DESCRIPTION OF THE DRAWING

FIG. 1: Transcytosis of NP and NP-Fc drug delivery systems using HUVEC as a model of endothelial cells. A monolayer of human umbelical vein cells (HUVEC) were grown on transwell forming tight junctions (TEER=˜170 Ω/cm²). Targeted (NP-Fc) and non-targeted (NP) nanoparticles were incubated on the apical side of transwells and collected from the basolateral side. The graph represents the percentage of nanoparticles that was collected from the basolateral side. Targeted nanoparticles are transported more efficiently (˜18%) than non-targeted nanoparticles (˜6%).

FIG. 2: Transcytosis of NP and NP-Fc drug delivery systems using Caco-2 as a model of epithelial cells. A monolayer of epithelial cells (Caco-2) were grown on transwell forming tight junctions (TEER=˜1000 Ω/cm²). Targeted (NP-Fc) and non-targeted (NP) nanoparticles were incubated on the apical side of transwells and collected from the basolateral side. The graph represents the percentage of nanoparticles that was collected from the basolateral side. Targeted nanoparticles are transported more efficiently (˜10%) than non-targeted nanoparticles (˜2%).

FIG. 3: Transcytosis of untargeted nanoparticles (NP) and nanoparticles targeted with IgG Fc ligand (NP-Fc) using wild type mice (Balb/c). Nanoparticles were fluorescently labeled for imaging. Five milligrams of nanoparticles with or without Fc in solution with protease inhibitors (250 μl) was gavaged into Balb/C mice (n=1). Duodenal tissues were collected 1 hour after gavage, fixed with para-formaldehyde, frozen into block and cryosectioned prior to fluorescent imaging. The fluorescent images represent the uptake of targeted and non-targeted fluorescent nanoparticles (red) 1 hour after gavage. The result shows that nanoparticles with Fc are targeting the intestine.

FIG. 4: Nanoparticle with Fc ligand conjugated to the surface. The copolymer is PLA (hydrophobic block) and PEG (hydrophilic block). Fc is conjugated to PEG as described in the methods section of Example 2.

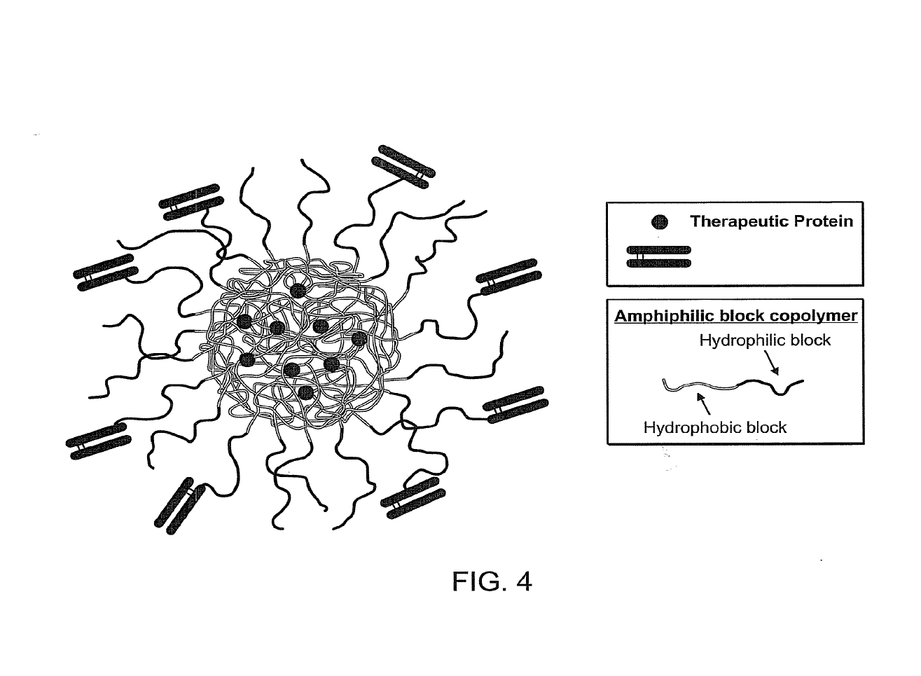

FIG. 5: Potential design for nanoparticle system with multiple surface functionalities. The two peptides shown here are the CREKA peptide (CREKA) for collagen IV binding and a MMP-2 degradable peptide (MMP-2). The polymer used may be PLA-PEG or PLGA-PEG.

### DEFINITIONS

The terms “angiogenesis inhibitor” and “anti-angiogenic agent” are used interchangeably herein to refer to agents that are capable of inhibiting or reducing one or more processes associated with angiogenesis including, but not limited to, endothelial cell proliferation, endothelial cell survival, endothelial cell migration, differentiation of precursor cells into endothelial cells, and capillary tube formation.

The term “animal,” as used herein, refers to any member of the animal kingdom. In some embodiments, “animal” refers to a human, at any stage of development. In some embodiments, “animal” refers to a non-human animal, at any stage of development. In some embodiments, animals include, but are not limited to, mammals, birds, reptiles, amphibians, fish, and/or worms. In certain embodiments, the non-human animal is a mammal (e.g., a rodent, a mouse, a rat, a rabbit, a monkey, a dog, a cat, a sheep, cattle, a primate, and/or a pig). In certain embodiments, a non-human animal is a domesticated animal. In some embodiments, an animal may be a transgenic animal, genetically-engineered animal, and/or clone.

The term “antibody,” as used herein, refers to an immunoglobulin, whether natural or wholly or partially synthetically produced. All derivatives thereof which maintain specific binding abilities are included in the term. The term covers any protein having a binding domain which is homologous or largely homologous to an immunoglobulin binding domain. In some embodiments, these proteins may be derived from natural sources. In some embodiments, these proteins are partly or wholly synthetically produced. An antibody may be a member of any immunoglobulin class (including any of the human classes: IgG, IgM, IgA, IgD, and IgE) and include any of the immunoglobulin isotypes. In some embodiments, derivatives of the IgG class and its isotypes are preferred in the present invention.

The term “antibody fragment,” as used herein, refers to any derivative of an antibody which is less than full-length. Examples of antibody fragments include, but are not limited to, Fab, Fab′, F(ab′)₂, scFv, Fv, dsFv diabody, Fc, and/or Fd fragments. In certain embodiments, a fragment is an Fc fragment, more particularly an Fc fragment of an IgG antibody. An antibody fragment may be produced by any means. For instance, in some embodiments, an antibody fragment may be enzymatically or chemically produced by fragmentation of an intact antibody. In some embodiments, an antibody fragment may be recombinantly produced from a gene encoding the partial antibody sequence. In some embodiments, an antibody fragment may be wholly or partially synthetically produced. An antibody fragment may optionally be a single chain antibody fragment. A functional antibody fragment typically comprises at least about 50 amino acids and more typically comprises at least about 200 amino acids.

The term “anti-infective agent,” as used herein, refers to any substance that inhibits the proliferation of one or more infectious agents, e.g., virus, bacteria, fungus, protozoa, helminth, fluke, or other parasite. The anti-infective agent may display inhibitory activity in vitro (i.e., in cell culture), in vivo (i.e., when administered to an animal at risk of or suffering from an infection), or both. Preferably the anti-infective agent has inhibitory activity in vivo at therapeutically tolerated doses. The anti-infective agent may be bacteriocidal or bacteriostatic.

The term “anti-inflammatory agent,” as used herein, refers to any substance that inhibits one or more signs or symptoms of inflammation.

The term “approximately,” as used herein, in reference to a number generally includes numbers that fall within a range of 5% or 10% in either direction of the number (greater than or less than the number) unless otherwise stated or otherwise evident from the context (except where such number would exceed 100% of a possible value).

The term “associated with,” as used herein, refers to the state of two or more entities which are linked by a direct or indirect covalent or non-covalent interaction. In some embodiments, an association is covalent. In some embodiments, a covalent association is mediated by a linker moiety. In some embodiments, an association is non-covalent (e.g., affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.). For example, in some embodiments, an agent to be delivered, an FcRn binding partner (e.g., Fc fragment), a nanoparticle, etc. may be covalently associated with one another. In some embodiments, an agent to be delivered, an FcRn binding partner (e.g., Fc fragment), a nanoparticle, etc. may be non-covalently associated with one another. For example, an agent to be delivered and an FcRn binding partner (e.g., Fc fragment) may be associated with the surface of, encapsulated within, surrounded by, and/or distributed throughout a polymeric matrix of a nanoparticle.

The term “biologically active agent,” as used herein, is any compound or agent, or its pharmaceutically acceptable salt, which possesses a desired biological activity, for example therapeutic, diagnostic and/or prophylactic properties in vivo. It is to be understood that the agent may need to be released from drug delivery systems for it to exert a biological activity. Biologically active agents include, but are not limited to, therapeutic agents as described herein. Biologically active agents may be, without limitation, artificial or naturally occurring small molecules (e.g., organic drugs, inorganic drugs, etc.), proteins (e.g., peptides, polypeptides, glycoproteins, etc.), immunoglobulins (e.g., antibodies), nucleic acids (e.g. vectors, RNAi-inducing entities, etc.), carbohydrates (e.g., monosaccharides, disaccharides, polysaccharides), lipids, cells (e.g. eukaryotic cells, prokaryotic cells, etc.), viruses, etc. In some embodiments, hormones, growth factors, drugs, cytokines, chemokines, clotting factors and endogenous clotting inhibitors, etc., are biologically active agents. For ease of reference, the term is also used to include detectable compounds, such as radiopaque compounds including air, barium, and/or magnetic compounds. A biologically active substance can be soluble or insoluble in water.

“Biocompatible” refers to substances that is substantially nontoxic to cells in the quantities and at the location used and/or does not elicit or cause a significant deleterious or untoward effect on the recipient's body at the location used, e.g., an unacceptable immunological or inflammatory reaction, unacceptable scar tissue formation, etc. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

“Biodegradable” means that a material is capable of being broken down physically and/or chemically within cells or within the body of a subject, e.g., by hydrolysis under physiological conditions and/or by natural biological processes such as the action of enzymes present within cells or within the body, and/or by processes such as dissolution, dispersion, etc., to form smaller chemical species which can typically be metabolized and, optionally, used by the body, and/or excreted or otherwise disposed of In some embodiments, a biodegradable compound is biocompatible. For purposes of the present invention, a polymer whose molecular weight decreases over time in vivo due to a reduction in the number of monomers is considered biodegradable.

The term “carrier” or “excipient,” as used herein, refers to one or more solid or liquid fillers, diluents, or encapsulating substances which are suitable for administration to a human, other mammal, or other animal. A “carrier” may be an organic or inorganic ingredient, natural or synthetic, with which a substance (e.g., an active ingredient) is combined to facilitate administration.

The term “homology,” as used herein, refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar. Both the identity and the approximate spacing of these amino acids relative to one another are considered for nucleotide sequences to be considered homologous.

The term “identity,” as used herein, refers to the overall relatedness between polymeric molecules, e.g. between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

The term “particle,” as used herein, refers to a small object, fragment, or piece of material and includes, without limitation, polymeric particles, biodegradable particles, non-biodegradable particles, single-emulsion particles, double-emulsion particles, coacervates, liposomes, microparticles, nanoparticles, macroscopic particles, pellets, crystals, aggregates, composites, pulverized, milled or otherwise disrupted matrices. Particles may be composed of a single substance or multiple substances. In certain embodiments of the invention a particle is not a viral particle. In certain embodiments of the invention, a particle is not a liposome. In some embodiments, a particle is an entity having a diameter of less than 10 microns (μm). Typically, particles have a longest dimension (e.g., diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In some embodiments, nanoparticles have a diameter of 50 nm or less. In some embodiments, nanoparticles have a diameter of 30 nm or less. In some embodiments, nanoparticles have a diameter of 20 nm or less. In some embodiments, nanoparticles have a diameter of 10 nm or less. In some embodiments, particles can be a matrix of polymers. In some embodiments, particles can be a non-polymeric particle (e.g., a metal particle, quantum dot, ceramic, inorganic material, bone, etc.).

The term “similarity,” as used herein, refers to the overall relatedness between polymeric molecules, e.g. between nucleic acid molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

The term “small molecule,” as used herein, is used to refer to molecules, whether naturally-occurring or artificially created (e.g., via chemical synthesis) that have a relatively low molecular weight. Typically, a small molecule is an organic compound (i.e., it contains carbon). A small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. In some embodiments, small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the U.S. Food and Drug Administration (U.S.F.D.A.) under 21 C.F.R. §§330.5, 331 through 361, and 440 through 460, incorporated herein by reference; drugs for veterinary use are listed by the U.S.F.D.A. under 21 C.F.R. §§500 through 589, incorporated herein by reference. All listed drugs are considered acceptable for use in accordance with the present invention.

The term “specific binding,” as used herein, refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. In some embodiments, binding of two or more entities may be considered specific if the equilibrium dissociation constant, K_{d}, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a K_{d} of greater than 10⁻³M). In some embodiments, specific binding, which can be referred to as “molecular recognition,” is a saturable binding interaction between two entities that is dependent on complementary orientation of functional groups on each entity. Examples of specific binding interactions include antibody-antigen interactions, aptamer-aptamer target interactions, avidin-biotin interactions, ligand-receptor interactions, metal-chelate interactions, hybridization between complementary nucleic acids, etc.

The term “target” or “marker,” as used herein, refers to any entity that is capable of specifically binding to a particular targeting moiety (e.g., FcRn binding partner, Fc fragment, etc.). In some embodiments, targets are specifically associated with one or more particular tissue types. In some embodiments, targets are specifically associated with one or more particular cell types. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid.

A substance is considered to be “targeted” for the purposes described herein if it specifically binds to a targeting moiety (e.g., FcRn binding partner, Fc fragment, etc.). In some embodiments, a targeting moiety (e.g., FcRn binding partner, Fc fragment, etc.) specifically binds to its target under stringent conditions. An inventive drug delivery conjugate comprising a targeting moiety (e.g., FcRn binding partner, Fc fragment, etc.) is considered to be “targeted” if the targeting moiety specifically binds to a target, thereby delivering the entire drug delivery conjugate composition to a specific organ, tissue, cell, and/or subcellular locale.

The term “targeting moiety,” as used herein, refers to any moiety that binds to a component associated with a cell. Such a component is referred to as a “target” or a “marker.” A targeting moiety may be a polypeptide, glycoprotein, nucleic acid, small molecule, carbohydrate, lipid, etc. In some embodiments, a targeting moiety is an antibody or characteristic portion thereof In some embodiments, a targeting moiety is an FcRn binding partner. In some embodiments, a targeting moiety is an Fc fragment.

The term “therapeutic agent” or “drug,” as used herein, refers to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (U.S.P.), Goodman and Gilman's The Pharmacological Basis of Therapeutics, 10^{th} Ed., McGraw Hill, 2001; Katzung, Ed., Basic and Clinical Pharmacology, McGraw-Hill/Appleton & Lange, 8^{th} ed., Sep. 21, 2000; Physician's Desk Reference (Thomson Publishing); and/or The Merck Manual of Diagnosis and Therapy, 18^{th} ed., 2006, Beers and Berkow, Eds., Merck Publishing Group; or, in the case of animals, The Merck Veterinary Manual, 9^{th} ed., Kahn, Ed., Merck Publishing Group, 2005; all of which are incorporated herein by reference.

The term “therapeutically effective amount,” as used herein, refers to an amount of a therapeutic, prophylactic, and/or diagnostic agent (e.g., inventive drug delivery conjugate) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

The term “treating,” as used herein, refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive drug delivery conjugate to a subject.

### DETAILED DESCRIPTION OF CERTAIN EMBODIMENTS OF THE INVENTION

The invention provides drug delivery systems comprising (1) a targeting moiety such as an FcRn binding partner (e.g., antibody fragment, Fc fragment, etc.) conjugated to an agent to be delivered, or (2) a drug delivery system (e.g., a polymeric particle, liposome, etc.) physically associated with a targeting moiety and an agent to be delivered; wherein the drug delivery system is capable of transferring its payload (e.g., therapeutic agent) across an endothelial and/or epithelial cell layer via a transcytosis mechanism. Expression of the FcRn surface protein on endothelial and/or epithelial cells offers a tool for targeted transport of a drug delivery system across a cell or cell layer. In certain embodiments, an IgG Fc fragment selectively binds to its receptor (FcRn) and induces the transport of the polymeric drug delivery system or agent across the cell layer for prophylactic, diagnostic, and/or therapeutic applications. An Fc fragment drug delivery system typically has a size ranging from about 1 nm to about 1000 nm. In some embodiments, an Fc fragment drug delivery system typically has a size ranging from about 10 nm to about 500 nm. In certain embodiments, particles are nanoparticles. In certain embodiments, particles are microparticles. In certain embodiments, particles are picoparticles.

### FcRn Receptor and Fc Fragments

The Fc receptor FcRn (n for neonatal) was first identified in 1970s as a protein that mediates transfer of maternal, milk born IgGs across the rodent neonatal intestine. More recent data have indicated that it not only delivers IgG across the maternofetal barrier during gestation, but is also responsible for the maintenance of serum IgGs level to provide humoral immunity during the first weeks of independent life. The IgG transfer is highly selective and is thought to involve specific receptors that bind to the Fc region of the IgG molecule.

The nature of FcRn and its interaction with IgGs involved in the transport of the IgG across subcell layer has been well characterized. In humans, maternal IgG is actively transported across the placenta. Several IgG-binding proteins have been isolated from placenta. FcγRII was detected in placental endothelium and FcγRIII in syncytiotrophoblasts. Both of these receptors, however, showed a relatively low affinity for monomeric IgG. The isolation from placenta of a cDNA encoding a human homolog of the rat and mouse enterocyte receptor for IgG has been reported (Story et al., 1994, J. Exp. Med., 180:2377; incorporated herein by reference). This Fc receptor for IgG may be responsible for the transport of maternal IgG to the human fetus (and even possibly to the neonate), as the molecule is highly homologous over its open reading frame with the rat FcRn sequence (69% nucleotide identity and 65% predicted amino acid identity). Placenta sections stained immunohistochemically with anti-hFcRn antibodies showed very low, occasional, or no expression of hFcRn in fetal endothelium. Other studies reported the expression of FcRn in functionally active form in mouse endothelial cells lines located within vesicular structures in the cytosol. FcRn has also been reported to be present in endothelial cells of human muscle vasculature. These data raised the question on the identity of the receptor or receptors that mediate the transfer of maternal IgG across the endothelial barrier. The isolation of human placental endothelial cells (HPEC) and the high expression of FcRn on these cells enabled the study of the transport of human IgG across cultured HPEC. In 2004, it was shown that in placental endothelial cells, FcRn is responsible for selective and controlled transport of IgG, as a result of which the fetal humoral immunity is ensured. These data add to the understanding of the basic mechanism for IgG traffic in human endothelial cells.

The Fc fragment has not yet been exploited as a delivery vehicle for therapeutic, diagnostic, prognostic, and prophylactic agents. Mucous membranes line the airways, the reproductive system, and the gastrointestinal tract, and this mucosal surface represents the first portal of entry for many diseases. An oral drug delivery system that is easy to administer and that triggers mucosal immunity would be highly desirable. In certain embodiments, the inventive drug delivery system is useful for delivering antigens to mucosal membranes for stimulating mucosal immunity. In certain embodiments, the inventive drug delivery system is useful for delivering proteins or peptides orally.

A conjugate of an Fc fragment and a controlled release system has not yet been demonstrated for these purposes. The present invention provides drug delivery systems based on Fc fragments and/or other targeting moieties that bind FcRn. Research has been focused only on the discovery that antigens or molecules that bind to the FcRn receptor, such as immunoglobulins, or portions thereof, are delivered across epithelial barriers by active transport through the enterocyte via FcRn receptors. The immunoglobulin or portion thereof binds to the FcRn receptor and acts as a carrier for the antigen as the immunoglobulin or portion thereof is transported across the epithelial barrier by FcRn mediated transport. The FcRn receptor is present in the human epithelial tissue of children and adults, and the invention therefore permits effective strategies for local, systemic, pulmonary, intravascular, and oral drug delivery systems in animals, particularly humans.

In some embodiments, after a drug delivery system comprising an FcRn binding partner (e.g., Fc fragment) is delivered to the interior of a cell, the FcRn binding partner (e.g., Fc fragment) can target the drug delivery system to immune system components (e.g. macrophages). In some cases, it may be desirable to target a drug delivery system to immune system components. In some embodiments, it may not be desirable to target a drug delivery system to immune system components. In some embodiments, drug delivery systems in accordance with the present invention are comprise FcRn binding partners that may be shed once the drug delivery system has reached its target. For example, an FcRn binding partner may be shed from the drug delivery system upon reaching the interior of a cell. In some embodiments, an FcRn binding partner may be shed from the drug delivery system upon reaching the cellular surface. In some embodiments, an FcRn binding partner may be shed from the drug delivery system after transcytosis. In some embodiments, an FcRn binding partner may be shed from the drug delivery system after it has reached the bloodstream. This shedding of the FcRn binding partner may be accomplished using a cleavable linker that associates the FcRn binding partner with the drug delivery system. Exemplary linkers useful in accordance with this embodiment include peptide linkers, esterase-sensitive linkers, disulfide linkers, and protease-sensitive linkers. In some embodiments, an FcRn binding partner may not be shed from the drug delivery system at any point during or after drug delivery.

In some embodiments, an FcRn binding partner may be associated with a drug delivery system (e.g. a particle or a conjugate) via a cleavable linker. For example, the linker may be a protease-cleavable linker. To give but one specific example, the linker may comprise the recognition sequence for matrix metalloproteinases (MMPs) that are typically either secreted into the extracellular space or bound to the external surface of a plasma membrane. When the drug delivery system reaches a cellular target, it is exposed to extracellular MMPs, which act upon the cleavable linker and shed the Fc fragment. Any kind of cleavable linker could be used in accordance with the invention, including, but not limited to, chemically-responsive linkers, pH-responsive linkers, heat-responsive linkers, light-responsive linkers (e.g., linkers that are cleaved in response to ultraviolet light), etc.

In some embodiments, a drug delivery system (e.g. a particle or a conjugate) may comprise targeting moieties in addition to the FcRn binding partner. Additional targeting moieties may help direct drug delivery systems to their appropriate targets. To give but one example, additional targeting moieties may target components of the extracellular matrix (ECM). In some embodiments, it may be desirable to target a drug delivery system to the ECM because it can minimize contact of the drug delivery system with cells of the immune system. For example, an additional targeting moiety may target collagen IV, one of the most abundant proteins of the basal lamina of the ECM. One of ordinary skill in the art will recognize that any additional targeting moiety which directs the drug delivery system to any target site may be utilized in accordance with the present invention. Exemplary additional targeting moieties include, but are not limited to, proteins (e.g., peptides, antibodies, glycoproteins, polypeptides, etc., or characteristic portions thereof), nucleic acids (e.g. aptamers, Spiegelmers, RNAi-inducing entities, etc., or characteristic portions thereof), carbohydrates (e.g. monosaccharides, disaccharides, polysaccharides, etc., or characteristic portions thereof), lipids or characteristic portions thereof, small molecules or characteristic portions thereof, viruses, nanoparticles, etc., as described herein.

An FcRn binding partner means any entity (e.g., peptides, glycopeptides, proteins, glycoproteins, polynucleotides, aptamers, spiegelmers, antibodies (e.g., monoclonal antibodies), antibody fragments, small molecule ligands, carbohydrate ligands, nanobodies, avimers, metal complexes, etc.) that can be specifically bound by the FcRn receptor with subsequent active transport by the FcRn receptor of the FcRn binding partner and its payload (e.g., particle or agent). As mentioned above, the FcRn receptor has been isolated from several mammalian species, including humans. The sequence of the human FcRn, rat FcRn, and mouse FcRn may be found in Story et al. (1994, J. Exp. Med., 180:2377; incorporated herein by reference). The FcRn receptor molecule is well characterized. The FcRn receptor binds IgG (but not other immunoglobulin classes such as IgA, IgD, IgM and IgE) at a relatively low pH, actively transports the IgG transcellularly in a luminal to serosal direction, and then releases the IgG at the relatively high pH found in the interstitial fluids. As will be recognized by those of ordinary skill in the art, FcRn receptors can be isolated by cloning or by affinity purification using, for example, monoclonal antibodies. Such isolated FcRn receptors then can be used to identify and isolate FcRn binding partners. The FcRn binding partner can be a small molecule, a protein or peptide, an immunoglobulin, a glycoprotein, a polynucleotide (e.g., aptamer, RNAi-inducing entity, etc.), a carbohydrate, a lipid, or any other type of chemical compound. In certain embodiments, the FcRn binding partner is a protein or peptide. In some embodiments, the FcRn binding partner is an immunoglobulin (e.g. Fc fragment). In some embodiments, it is an aptamer. In some embodiments, it is a spiegelmer. In some embodiments, it is an RNAi-inducing entity (e.g., siRNA, shRNA, miRNA, etc.). In some embodiments, the binding partner is a small molecule.

In certain embodiments, an FcRn binding partner is an Fc fragment. In certain embodiments, an FcRn binding partner is an Fc fragment of an IgG antibody. In some embodiments, an FcRn binding partner is an Fc fragment of any isotype of IgG antibody (e.g., IgG 1, IgG 2, IgG 2a, IgG 2b, IgG 3, IgG 4, etc.).

In some embodiments, the sequence of the Fc portion of a human IgG 1 antibody is as follows:

(SEQ ID NO.: 1)
TCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKG
FYPSDIAVEWESNGQPENNYKTTPPVLDSDGPFFLYSKLTVDKSRWQQG
NVFSCSVMHEALHNHYTQKSLSLSPGK.

In some embodiments, the sequence of the Fc portion of a human IgG 1 antibody is as follows:

(SEQ ID NO.: 2)
ZVQLEQSGPGLVRPSQTLSLTCTVSGTSFDDYYWTWVRQPPGRGLEWIG
YVFYTGTTLLDPSLRGRVTMLVNTSKNQFSLRLSSVTAADTAVYYCARN
LIAGGIDVWGQGSLVTVSSASTKGPSVFPLAPSSKSTSGGTAALGCLVK
DYFPEPVTVSWNSGALTSGVHTFPAVLQSSGLYSLSSVVTVPSSSLGTQ
TYICNVNHKPSNTKVDKKVEPKSC

In some embodiments, the nucleic acid sequence corresponding to the Fc portion of a human IgG 1 antibody is as follows:

(SEQ ID NO.: 3)
GACAAAACTCACACATGCCCACCGTGCCCAGCACCTGAACTCCTGGGGG
GACCGTCAGTCTTCCTCTTCCCCCCAAAACCCAAGGACACCCTCATGAT
CTCCCGGACCCCTGAGGTCACATGCGTGGTGGTGGACGTGAGCCACGAA
GACCCTGAGGTCAAGTTCAACTGGTACGTGGACGGCGTGGAGGTGCATA
ATGCCAAGACAAAGCCGCGGGAGGAGCAGTACAACAGCACGTACCGTGT
GGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAA
CCATCTCCAAAGCCAAAGGGCAGCCCCGAGAACCACAGGTGTACACCCT
GCCCCCATCCCGGGAGGAGATGACCAAGAACCAGGTCAGCCTGACCTGC
CTGGTCAAAGGCTTCTATCCCAGCGACATCGCCGTGGAGTGGGAGAGCA
ATGGGCAGCCGGAGAACAACTACAAGACCACGCCTCCCGTGCTGGACTC
CGACGGCTCCTTCTTCCTCTACAGCAAGCTCACCGTGGACAAGAGCAGG
TGGCAGCAGGGGAACGTCTTCTCATGCTCCGTGATGCATGAGGGTCTGC
ACAACCACTACACGCAGAAGAGCCTCTCCCTGTCTCCGGGTAAATGA.

In some embodiments, the nucleic acid sequence corresponding to the Fc portion of a human IgG 2 antibody is as follows:

(SEQ ID NO.: 4)
GTGGAGTGCCCACCTTGCCCAGCACCACCTGTGGCAGGACCTTCAGTCT
TCCTCTTCCCCCCAAAACCCAAGGACACCCTGATGATCTCCAGAACCCC
TGAGGTCACGTGCGTGGTGGTGGACGTGAGCCACGAAGACCCCGAGGTC
CAGTTCAACTGGTACGTGGACGGCATGGAGGTGCATAATGCCAAGACAA
AGCCACGGGAGGAGCAGTTCAACAGCACGTTCCGTGTGGTCAGCGTCCT
CACCGTCGTGCACCAGGACTGGCTGAACGGCAAGGAGTACAAGTGCAAG
GTCTCCAACAAAGGCCTCCCAGCCCCCATCGAGAAAACCATCTCCAAAA
CCAAAGGGCAGCCCCGAGAACCACAGGTGTACACCCTGCCCCCATCCCG
GGAGGAGATGACCAAGAACCAGGTCAGCCTGACCTGCCTGGTCAAAGGC
TTCTACCCCAGCGACATCGCCGTGGAGTGGGAGAGCAATGGGCAGCCGG
AGAACAACTACAAGACCACACCTCCCATGCTGGACTCCGACGGCTCCTT
CTTCCTCTACAGCAAGCTCACCGTGGACAAGAGCAGGTGGCAGCAGGGG
AACGTCTTCTCATGCTCCGTGATGCATGAGGCTCTGCACAACCACTACA
CACAGAAGAGCCTCTCCCTGTCTCCGGGTAAATGAGTGCCACGGCTAGC
TGG.

In some embodiments, the nucleic acid sequence corresponding to the Fc portion of a human IgG 3 antibody is as follows:

(SEQ ID NO.: 5)
GACACACCTCCCCCGTGCCCAAGGTGCCCAGCACCTGAACTCCTGGGAG
GACCGTCAGTCTTCCTCTTCCCCCCAAAACCCAAGGATACCCTTATGAT
TTCCCGGACCCCTGAGGTCACGTGCGTGGTGGTGGACGTGAGCCACGAA
GACCCCGAGGTCCAGTTCAAGTGGTACGTGGACGGCGTGGAGGTGCATA
ATGCCAAGACAAAGCCGCGGGAGGAGCAGTTCAACAGCACGTTCCGTGT
GGTCAGCGTCCTCACCGTCCTGCACCAGGACTGGCTGAACGGCAAGGAG
TACAAGTGCAAGGTCTCCAACAAAGCCCTCCCAGCCCCCATCGAGAAAA
CCATCTCCAAAACCAAAGGACAGCCCCGAGAACCACAGGTGTACACCCT
GCCCCCATCCCGGGAGGAGATGACCAAGAACCAGGTCAGCCTGACCTGC
CTGGTCAAAGGCTTCTACCCCAGCGACATCGCCGTGGAGTGGGAGAGCA
GCGGGCAGCCGGAGAACAACTACAACACCACGCCTCCCATGCTGGACTC
CGACGGCTCCTTCTTCCTCTACAGCAAGCTCACCGTGGACAAGAGCAGG
TGGCAGCAGGGGAACATCTTCTCATGCTCCGTGATGCATGAGGCTCTGC
ACAACCGCTTCACGCAGAAGAGCCTCTCCCTGTCTCCGGGTAAATGA.

In some embodiments, the nucleic acid sequence corresponding to the Fc portion of a human IgG 4 antibody is as follows:

(SEQ ID NO.: 6)
CCCCCATGCCCATCATGCCCAGCACCTGAGTTCCTGGGGGGACCATCAG
TCTTCCTGTTCCCCCCAAAACCCAAGGACACTCTCATGATCTCCCGGAC
CCCTGAGGTCACGTGCGTGGTGGTGGACGTGAGCCAGGAAGACCCCGAG
GTCCAGTTCAACTGGTACGTGGATGGCGTGGAGGTGCATAATGCCAAGA
CAAAGCCGCGGGAGGAGCAGTTCAACAGCACGTACCGTGTGGTCAGCGT
CCTCACCGTCCTGCACCAGGACTGGCTGAACGGCAAGGAGTACAAGTGC
AAGGTCTCCAACAAAGGCCTCCCGTCCTCCATCGAGAAAACCATCTCCA
AAGCCAAAGGGCAGCCCCGAGAGCCACAGGTGTACACCCTGCCCCCATC
CCAGGAGGAGATGACCAAGAACCAGGTCAGCCTGACCTGCCTGGTCAAA
GGCTTCTACCCCAGCGACATCGCCGTGGAGTGGGAGAGCAATGGGCAGC
CGGAGAACAACTACAAGACCACGCCTCCCGTGCTGGACTCCGACGGCTC
CTTCTTCCTCTACAGCAGGCTAACCGTGGACAAGAGCAGGTGGCAGGAG
GGGAATGTCTTCTCATGCTCCGTGATGCATGAGGCTCTGCACAACCACT
ACACACAGAAGAGCCTCTCCCTGTCTCCGGGTAAATG.

An FcRn binding partner is at least 50% homologous to any sequence of an Fc portion of any IgG antibody. In some embodiments, an FcRn binding partner is at least 60% homologous to any sequence of an Fc portion of any IgG antibody. In certain embodiments, an FcRn binding partner is at least 70% homologous to any sequence of an Fc portion of any IgG antibody. In certain embodiments, an FcRn binding partner is at least 80% homologous to any sequence of an Fc portion of any IgG antibody. In certain embodiments, an FcRn binding partner is at least 90% homologous to any sequence of an Fc portion of any IgG antibody. In certain embodiments, an FcRn binding partner is at least 95% or at least 98% homologous to any sequence of an Fc portion of any IgG antibody.

An FcRn binding partner is at least 50% homologous to any of SEQ ID NOs.: 1-6. In certain embodiments, an FcRn binding partner is at least 60% homologous to any of SEQ ID NOs.: 1-6. In certain embodiments, an FcRn binding partner is at least 70% homologous to any of SEQ ID NOs.: 1-6. In certain embodiments, an FcRn binding partner is at least 80% homologous to any of SEQ ID NOs.: 1-6. In certain embodiments, an FcRn binding partner is at least 90% homologous to any of SEQ ID NOs.: 1-6. In certain embodiments, an FcRn binding partner is at least 95% or at least 98% homologous to any of SEQ ID NOs.: 1-6.

In some embodiments, an FcRn binding partner may comprise any portion of the Fc fragment of any IgG isotype. In specific embodiments, the portion of the Fc fragment retains the ability to bind the FcRn receptor. In some embodiments, an FcRn binding partner may be any substance that is able to specifically bind to the FcRn receptor. In some embodiments, an FcRn binding partner is any substance that is able to bind to the FcRn receptor with an equilibrium dissociation constant, K_{d}, that is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed.

In some embodiments, an FcRn binding partner may be modified such that it is less immunogenic than the unmodified FcRn binding partner. In some embodiments, an Fc binding partner may be modified such that it does not bind to complement systems.

In some embodiments, an FcRn binding partner may comprise any portion of the Fc fragment of any IgG isotype derived from any animal source. In some embodiments, a human Fc sequence may be used. In some embodiments, an Fc fragment may be derived from any animal, such as a rodent, a mouse, a rat, a rabbit, a monkey, a dog, a cat, a primate, a pig, etc.

In some embodiments, an FcRn binding partner comprises at least about 5 amino acids, at least about 10 amino acids, at least about 20 amino acids, at least about 30 amino acids, at least about 40 amino acids, at least about 50 amino acids, at least about 75 amino acids, at least about 100 amino acids, at least about 125 amino acids, at least about 150 amino acids, at least about 175 amino acids, or at least about 200 amino acids of any of SEQ ID NOs.: 1-6.

In some embodiments, an RcRn binding partner comprises at least about 5 amino acids, at least about 10 amino acids, at least about 20 amino acids, at least about 30 amino acids, at least about 40 amino acids, at least about 50 amino acids, at least about 75 amino acids, at least about 100 amino acids, at least about 125 amino acids, at least about 150 amino acids, at least about 175 amino acids, or at least about 200 amino acids that are at least about 50% homologous to any of SEQ ID NOs.: 1-6.

In some embodiments, an RcRn binding partner comprises at least about 5 amino acids, at least about 10 amino acids, at least about 20 amino acids, at least about 30 amino acids, at least about 40 amino acids, at least about 50 amino acids, at least about 75 amino acids, at least about 100 amino acids, at least about 125 amino acids, at least about 150 amino acids, at least about 175 amino acids, or at least about 200 amino acids that are at least about 80% homologous to any of SEQ ID NOs.: 1-6.

In some embodiments, an RcRn binding partner comprises at least about 5 amino acids, at least about 10 amino acids, at least about 20 amino acids, at least about 30 amino acids, at least about 40 amino acids, at least about 50 amino acids, at least about 75 amino acids, at least about 100 amino acids, at least about 125 amino acids, at least about 150 amino acids, at least about 175 amino acids, or at least about 200 amino acids that are at least about 90% homologous to any of SEQ ID NOs.: 1-6.

In some embodiments, an RcRn binding partner comprises at least about 5 amino acids, at least about 10 amino acids, at least about 20 amino acids, at least about 30 amino acids, at least about 40 amino acids, at least about 50 amino acids, at least about 75 amino acids, at least about 100 amino acids, at least about 125 amino acids, at least about 150 amino acids, at least about 175 amino acids, or at least about 200 amino acids that are at least about 95% homologous to SEQ ID NO.: 1.

In certain embodiments, rather than using an FcRn binding partner, the polymeric drug delivery system is conjugated to a binding partner of another receptor (i.e., a non-FcRn receptor) found on endothelial or epithelial cells. In certain particular embodiments, the non-FcRn receptor is found on endothelial cells. In some embodiments, the non-FcRn receptor is found on epithelial cells. In certain embodiments, the polymeric drug delivery system is conjugated not only to an FcRn binding partner but also a binding partner for another receptor found on endothelial or epithelial cells. In certain embodiments, the binding partner of a non-FcRn receptor is a binding partner of an adhesion molecule (e.g., selectins, integrins, immunoglobulin superfamily, cadherins, etc.). For a list of adhesion molecules, see, e.g., Carlos et al. (1994, Blood, 84:2068; incorporated herein by reference). In certain embodiments, the other binding partner is a binding partner of a member of the immunoglobulin superfamily (e.g., NCAM-1; ICAM-1; ICAM-2; LFA-3; major histocompatibility complex molecules (MHCs), particular class I MHC; PECAM (CD31); VCAM-1; MAdCAM-1; PECAM-1). In certain embodiments, the other binding partner is a binding partner of vascular cell adhesion molecule (e.g., VCAM-1). In certain specific embodiments, the polymeric drug delivery system has conjugated to it an Fc fragment and a binding partner of VCAM-1. In certain embodiments, the other binding partner is a binding partner of intercellular adhesion molecule (e.g., ICAM-1, ICAM-2). In certain specific embodiments, the polymeric drug delivery system has conjugated to it an Fc fragment and a binding partner of an ICAM receptor (e.g., ICAM-1, ICAM-2). In some embodiments, the other binding partner is a binding partner of selectin (e.g., E-selectin, P-selectin, L-selectin). In certain specific embodiments, the polymeric drug delivery system has conjugated to it an Fc fragment and a binding partner of selectin (e.g., E-selectin, P-selectin, L-selectin). In some embodiments, the other binding partner is a binding partner of a member of the integrin family (e.g., α1β1, α2β1, α3β1, α4β1, α5β1, α6β1, α7β1, α8β1, α9β1, αvβ3, α6β4, αvβ5). In certain embodiments, the polymeric drug delivery system has conjugated to it an Fc fragment and a binding partner of a member of the integrin family (e.g., α1β1, α2β1, α3β1, α4β1, α5β1, α6β1, α7β1, α8β1, α9β1, αvβ3, α6β4, αvβ5). In some embodiments, the other binding partner is a binding partner of a member of the cadherin family (e.g., cadherin E, cadherin P, cadherin VE (CD144), desmocollin 2, desmoglein 2, etc.). In certain embodiments, the polymeric drug delivery system has conjugated to it an Fc fragment and a binding partner of a member of the cadherin family (e.g., cadherin E, cadherin P, cadherin VE (CD144), desmocollin 2, desmoglein 2, etc.). In some embodiments, the other binding partner is a binding partner of a member of the adressin family, particularly vascular addressins (e.g., PNAd, Mad, GlyCAM-1, CD34, MAdCAM-1, etc.). In certain embodiments, the polymeric drug delivery system has conjugated to it an Fc fragment and a binding partner of a member of the cadherin family (e.g., PNAd, Mad, GlyCAM-1, CD34, MAdCAM-1, etc.). In some embodiments, the other binding partner is a binding partner of other adhesion molecules. In certain embodiments, the polymeric drug delivery system has conjugated to it an Fc fragment and a binding partner of other adhesion molecules. Combinations of various binding partners of the above-described receptors may be used on an inventive polymeric delivery system. In certain embodiments, one, two, three, four, or five binding partners are used on an inventive particle. In some embodiments, a combination including more than five binding partners is used.

### Particles

In certain embodiments, the present invention provides a drug delivery system comprising an Fc fragment physically associated with a particle. In certain embodiments, a drug delivery system, as used herein, comprises a particle (e.g., particle comprising a polymeric matrix; non-polymeric particle; etc.) associated with an active agent to be delivered, such as a therapeutic agent, a diagnostic agent, a prognostic agent, and/or prophylactic agent, so that the active agent is released from the particle.

In certain embodiments, the controlled release polymer system comprises a particle. Any particle can be used in accordance with the present invention. In some embodiments, particles are biodegradable and biocompatible. In general, a biocompatible substance is not toxic to cells. In some embodiments, a substance is considered to be biocompatible if its addition to cells results in less than a certain threshhold of cell death. In some embodiments, a substance is considered to be biocompatible if its addition to cells does not induce adverse effects. In general, a biodegradable substance is one that undergoes breakdown under physiological conditions over the course of a therapeutically relevant time period (e.g., weeks, months, or years). In some embodiments, a biodegradable substance is a substance that can be broken down by cellular machinery. In some embodiments, a biodegradable substance is a substance that can be broken down by chemical processes. In some embodiments, a particle is a substance that is both biocompatible and biodegradable. In some embodiments, a particle is a substance that is biocompatible, but not biodegradable. In some embodiments, a particle is a substance that is biodegradable, but not biocompatible.

An agent to be delivered may be released by diffusion, dissolution, degradation of the polymer, or a combination thereof. In some embodiments, biodegradable polymers degrade by bulk erosion. In some embodiments, biodegradable polymers degrade by surface erosion.

In some embodiments, a particle which is biocompatible and/or biodegradable may be associated with a therapeutic, diagnostic, and/or prophylactic agent that is not biocompatible, is not biodegradable, or is neither biocompatible nor biodegradable (e.g., a cytotoxic agent). In some embodiments, a particle which is biocompatible and/or biodegradable may be associated with a therapeutic, diagnostic, and/or prophylactic agent that is also biocompatible and/or biodegradable.

In general, a particle in accordance with the present invention is any entity having a greatest dimension (e.g., diameter) of less than 100 microns (μm). In some embodiments, inventive particles have a greatest dimension of less than 10 μm. In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 300 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

In some embodiments, particles have a diameter of approximately 1000 nm. In some embodiments, particles have a diameter of approximately 750 nm. In some embodiments, particles have a diameter of approximately 500 nm. In some embodiments, particles have a diameter of approximately 450 nm. In some embodiments, particles have a diameter of approximately 400 nm. In some embodiments, particles have a diameter of approximately 350 nm. In some embodiments, particles have a diameter of approximately 300 nm. In some embodiments, particles have a diameter of approximately 275 nm. In some embodiments, particles have a diameter of approximately 250 nm. In some embodiments, particles have a diameter of approximately 225 nm. In some embodiments, particles have a diameter of approximately 200 nm. In some embodiments, particles have a diameter of approximately 175 nm. In some embodiments, particles have a diameter of approximately 150 nm. In some embodiments, particles have a diameter of approximately 125 nm. In some embodiments, particles have a diameter of approximately 100 nm. In some embodiments, particles have a diameter of approximately 75 nm. In some embodiments, particles have a diameter of approximately 50 nm In some embodiments, particles have a diameter of approximately 25 nm.

In some embodiments, populations of particles are characterized by a mean particle diameter. In some embodiments, mean particle diameter is measured by particle weight. In some embodiments, mean particle diameter is measured by the total number of particles. In some embodiments, the diameter of no more than 1% of particles varies from the mean particle diameter by more than 150% of the mean particle diameter. In some embodiments, the diameter of no more than 1% of particles varies from the mean particle diameter by more than 100% of the mean particle diameter. In some embodiments, the diameter of no more than 1% of particles varies from the mean particle diameter by more than 75% of the mean particle diameter. In some embodiments, the diameter of no more than 1% of particles varies from the mean particle diameter by more than 50% of the mean particle diameter. In some embodiments, the diameter of no more than 1% of particles varies from the mean particle diamter by more than 25% of the mean particle diameter.

In some embodiments, no more than 5% of particles varies by more than 150% of the mean particle diameter. In some embodiments, no more than 5% of particles varies by more than 100% of the mean particle diameter. In some embodiments, no more than 5% of particles varies by more than 75% of the mean particle diameter. In some embodiments, no more than 5% of particles varies by more than 50% of the mean particle diameter. In some embodiments, no more than 5% of particles varies by more than 25% of the mean particle diameter.

In some embodiments, no more than 10% of particles varies by more than 150% of the mean particle diameter. In some embodiments, no more than 10% of particles varies by more than 100% of the mean particle diameter. In some embodiments, no more than 10% of particles varies by more than 75% of the mean particle diameter. In some embodiments, no more than 10% of particles varies by more than 50% of the mean particle diameter. In some embodiments, no more than 10% of particles varies by more than 25% of the mean particle diameter.

In some embodiments, no more than 25% of particles varies by more than 150% of the mean particle diameter. In some embodiments, no more than 25% of particles varies by more than 100% of the mean particle diameter. In some embodiments, no more than 25% of particles varies by more than 75% of the mean particle diameter. In certain embodiments, no more than 25% of particles varies by more than 50% of the mean particle diameter. In some embodiments, no more than 25% of particles varies by more than 25% of the mean particle diameter.

In some embodiments, the diameter of any individual particle varies by no more than 25% of the mean particle diameter of the particle population. In some embodiments, the diameter of any individual particle varies by no more than 50% of the mean particle diameter of the particle population. In some embodiments, the diameter of any individual particle varies by no more than 75% of the mean particle diameter of the particle population. In some embodiments, the diameter of any individual particle varies by no more than 100% of the mean particle diameter of the particle population. In some embodiments, the diameter of any individual particle varies by no more than 150% of the mean particle diameter of the particle population. In some embodiments, the diameter of any individual particle varies by no more than 200% of the mean particle diameter of the particle population.

In some embodiments, the diameter of approximately 10% of the particles varies by no more than 50% above the mean particle diameter, and wherein the diameter of approximately 10% of the particles varies by no more than 50% below the mean particle diameter. In some embodiments, the diameter of approximately 10% of the particles varies by no more than 25% above the mean particle diameter, and wherein the diameter of approximately 10% of the particles varies by no more than 25% below the mean particle diameter. In some embodiments, the diameter of approximately 10% of the particles varies by no more than 10% above the mean particle diameter, and wherein the diameter of approximately 10% of the particles varies by no more than 10% below the mean particle diameter.

In certain embodiments, particles are greater in size than the renal excretion limit (e.g. particles having diameters of greater than 6 nm). In specific embodiments, particles have diameters greater than 5 nm, greater than 10 nm, greater than 15 nm, greater than 20 nm, greater than 50 nm, greater than 100 nm, greater than 250 nm, greater than 500 nm, greater than 1000 nm, or larger. In certain embodiments, particles are small enough to avoid clearance of particles from the bloodstream by the liver (e.g. particles having diameters of less than 1000 nm). In specific embodiments, particles have diameters less than 1500 nm, less than 1000 nm, less than 750 nm, less than 500 nm, less than 250 nm, less than 100 nm, or smaller. In general, physiochemical features of particles, including particle size, can be selected to allow a particle to circulate longer in plasma by decreasing renal excretion and/or liver clearance. In some embodiments, particles have diameters ranging from 5 nm to 1500 nm, from 5 nm to 1000 nm, from 5 nm to 750 nm, from 5 nm to 500 nm, from 5 nm to 250 nm, or from 5 nm to 100 nm. In some embodiments, produced particles have diameters ranging from 10 nm to 1500 nm, from 15 nm to 1500 nm, from 20 nm to 1500 nm, from 50 nm to 1500 nm, from 100 nm to 1500 nm, from 250 nm to 1500 nm, from 500 nm to 1500 nm, or from 1000 nm to 1500 nm.

It is often desirable to utilize a population of particles that is relatively uniform in terms of size, shape, and/or composition so that each particle has similar properties. For example, at least 80%, at least 90%, or at least 95% of the particles of a population of particles may have a diameter or greatest dimension that falls within 5%, 10%, or 20% of the average diameter or greatest dimension. In some embodiments, a population of particles may be heterogeneous with respect to size, shape, and/or composition.

Zeta potential is a measurement of surface potential of a particle. In some embodiments, particles have a zeta potential ranging between −50 mV and +50 mV. In some embodiments, particles have a zeta potential ranging between −25 mV and +25 mV. In some embodiments, particles have a zeta potential ranging between −10 mV and +10 mV. In some embodiments, particles have a zeta potential ranging between −5 mV and +5 mV. In some embodiments, particles have a zeta potential ranging between 0 mV and +50 mV. In some embodiments, particles have a zeta potential ranging between 0 mV and +25 mV. In some embodiments, particles have a zeta potential ranging between 0 mV and +10 mV. In some embodiments, particles have a zeta potential ranging between 0 mV and +5 mV. In some embodiments, particles have a zeta potential ranging between −50 mV and 0 mV. In some embodiments, particles have a zeta potential ranging between −25 mV and 0 mV. In some embodiments, particles have a zeta potential ranging between −10 mV and 0 mV. In some embodiments, particles have a zeta potential ranging between −5 mV and 0 mV. In some embodiments, particles have a substantially neutral zeta potential (i.e., approximately 0 mV).

A variety of different particles can be used in accordance with the present invention. In some embodiments, particles are spheres or spheroids. In some embodiments, particles are flat or plate-shaped. In some embodiments, particles are cubes or cuboids. In some embodiments, particles are ovals or ellipses. In some embodiments, particles are cylinders, cones, or pyramids.

In some embodiments, particles are microparticles (e.g., microspheres). In general, a “microparticle” refers to any particle having a diameter of less than 1000 μm. In some embodiments, particles are nanoparticles (e.g., nanospheres). In general, a “nanoparticle” refers to any particle having a diameter of less than 1000 nm. In some embodiments, particles are picoparticles (e.g., picospheres). In general, a “picoparticle” refers to any particle having a diameter of less than 1 nm. In some embodiments, particles are liposomes. In some embodiments, particles are micelles.

Particles can be solid or hollow and can comprise one or more layers (e.g., nanoshells, nanorings). In some embodiments, each layer has a unique composition and unique properties relative to the other layer(s). To give but one example, particles may have a core/shell structure, wherein the core is one layer and the shell is a second layer. Particles may comprise a plurality of different layers. In some embodiments, one layer may be substantially cross-linked, a second layer is not substantially cross-linked, and so forth. In some embodiments, one, a few, or all of the different layers may comprise one or more agents to be delivered (e.g., therapeutic, diagnostic, and/or prophylactic agents). In some embodiments, one layer comprises an agent to be delivered, a second layer does not comprise an agent to be delivered, and so forth. In some embodiments, each individual layer comprises a different agent or set of agents to be delivered.

In certain embodiments of the invention, a particle is porous, by which is meant that the particle contains holes or channels, which are typically small compared with the size of a particle. For example a particle may be a porous silica particle, e.g., a mesoporous silica nanoparticle or may have a coating of mesoporous silica (Lin et al., 2005, J. Am. Chem. Soc., 17:4570). Particles may have pores ranging from about 1 nm to about 50 nm in diameter, e.g., between about 1 and 20 nm in diameter. Between about 10% and 95% of the volume of a particle may consist of voids within the pores or channels.

Particles may have a coating layer. Use of a biocompatible coating layer can be advantageous, e.g., if the particles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, etc. In some embodiments, a suitable coating layer is PEG. In some embodiments, a suitable coating layer is a PEG copolymer. Coatings may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

In some embodiments, particles may optionally comprise one or more dispersion media, surfactants, release-retarding ingredients, or other pharmaceutically acceptable excipient. In some embodiments, particles may optionally comprise one or more plasticizers or additives.

### Particles Comprising a Polymeric Matrix

In some embodiments, particles comprise a matrix of polymers. In some embodiments, a therapeutic, diagnostic, and/or prophylactic agent and FcRn binding partner (e.g., Fc fragment) is covalently associated with a polymeric matrix. In some embodiments, covalent association is mediated by a linker (e.g., an aliphatic or heteroaliphatic linker). In some embodiments, a therapeutic, diagnostic, and/or prophylactic agent and FcRn binding partner (e.g., Fc fragment) is non-covalently associated with a polymeric matrix. In some embodiments, a therapeutic, diagnostic, and/or prophylactic agent and FcRn binding partner (e.g., Fc fragment) is associated with the surface of, encapsulated within, surrounded by, and/or dispersed throughout a polymeric matrix.

A wide variety of polymers and methods for forming particles therefrom are known in the art of drug delivery. In some embodiments of the invention, the matrix of a particle comprises one or more polymers. Any polymer may be used in accordance with the present invention. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers include polyalkylenes (e.g., polyethylenes), polycarbonates (e.g., poly(1,3-dioxan-2one)), polyanhydrides (e.g., poly(sebacic anhydride)), polyhydroxyacids (e.g., poly(β-hydroxyalkanoate)), polyfumarates, polycaprolactones, polyamides (e.g., polycaprolactam), polyacetals, polyethers, polyesters (e.g., polylactide, polyglycolide), poly(orthoesters), polyvinyl alcohols, polyurethanes, polyphosphazenes, polyacrylates, polymethacrylates, polycyanoacrylates, polyureas, polystyrenes, polyamines, poly(arylates), polycarbonates, poly(propylene fumarates), polyhydroxyalkanoates, polyketals, polyesteramides, poly(dioxanones), polyhydroxybutyrates, polyhydroxyvalyrates, polyorthocarbonates, poly(vinyl pyrrolidone), polyalkylene oxalates, polyalkylene succinates, poly(malic acid), poly(methyl vinyl ether), and poly(maleic anhydride). In some embodiments, polymers in accordance with the present invention include polymers which have been approved for use in humans by the United States Food and Drug Administration (U.S.F.D.A.) under 21 C.F.R. §177.2600, including but not limited to polyesters (e.g., polylactic acid, polyglycolic acid, poly(lactic-co-glycolic acid)), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group).

In some embodiments, polymers may be modified with one or more moieties and/or functional groups. Any moiety or functional group can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). In some embodiments, polymers may be modified with PEG.

In some embodiments, polymers may be modified with a lipid or fatty acid group, properties of which are described in further detail below. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; lactide-PEG copolymers (e.g., PLA-PEG copolymers); glycolide-PEG copolymers (e.g., PGA-PEG copolymers); copolymers of lactide and glycolide (e.g., PLGA); copolymers of lactide, glycolide, and PEG (e.g., PLGA-PEG copolymers); and derivatives thereof In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethylene imine), poly(ethylene imine)-PEG copolymers, poly(L-lactide-co-L-lysine), poly(serine ester), poly(4-hydroxy-L-proline ester), poly[α-(4-aminobutyl)-L-glycolic acid], and derivatives thereof.

In certain embodiments, a polymer may be PLA. In certain embodiments, a polymer may be PGA. In certain embodiments, a polymer may be PLGA. In certain embodiments, a polymer may be PEG. In certain embodiments, a polymer may be PEG-PLA. In certain embodiments, a polymer may be PEG-PGA. In certain embodiments, a polymer may be PEG-PLGA. In certain embodiments, a polymer may be a PEG-PLA/PLA blend. In certain embodiments, a polymer may be a PEG-PGA/PGA blend. In certain embodiments, a polymer may be a PEG-PLGA/PEG-PLGA blend. In certain embodiments, a polymer may be a PEG-PLA/PGA blend. In certain embodiments, a polymer may be PEG-PLA/PLGA blend. In certain embodiments, a polymer may be a PEG-PGA/PLA blend. In certain embodiments, a polymer may be a PEG-PLGA/PLA blend. In certain embodiments, a polymer may be a PEG-PLGA/PGA blend. In certain embodiments, a polymer may be a PEG-PGA/PLGA blend. In certain embodiments, any of the foregoing may comprise a modified PEG (e.g. methoxy(polyethylene glycol)). For example, a polymer may be methoxy(polyethylene glycol)-PLA. In some embodiments, a polymer may comprise any combination or blend of the foregoing.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 65:35, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g., DNA, RNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633). Poly(4-hydroxy-L-proline ester) was recently demonstrated to condense plasmid DNA through electrostatic interactions, and to mediate gene transfer (Putnam et al., 1999, Macromolecules, 32:3658; and Lim et al., 1999, J. Am. Chem. Soc., 121:5633). These new polymers are less toxic than poly(lysine) and PEI, and they degrade into non-toxic metabolites.

In some embodiments, polymers can be anionic polymers. In some embodiments, anionic polymers comprise carboxyl, sulfate, or groups. To give but a few examples, anionic polymers include, but are not limited to, dextran sulfate, heparan sulfate, alginic acid, polyvinylcarboxylic acid, arabic acid carboxymethylcellulose, and the like. In some embodiments, anionic polymers are provided as a salt (e.g., sodium salt).

In some embodiments, a polymer in accordance with the present invention may be a carbohydrate, properties of which are described in further detail below. In some embodiments, a carbohydrate may be a polysaccharide comprising simple sugars (or their derivatives) connected by glycosidic bonds, as known in the art. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan.

In some embodiments, a polymer in accordance with the present invention may be a protein or peptide, properties of which are described in further detail below. Exemplary proteins that may be used in accordance with the present invention include, but are not limited to, albumin, collagen, gelatin, a poly(amino acid) (e.g., polylysine), an antibody, etc.

In some embodiments, a polymer in accordance with the present invention may be a nucleic acid (i.e., polynucleotide), properties of which are described in further detail below. Exemplary polynucleotides that may be used in accordance with the present invention include, but are not limited to, DNA, RNA, etc.

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step.

It is further to be understood that controlled release polymer systems may be a homopolymer, block copolymer, diblock triblock, multibock copolymer, linear polymer, dendritic polymer, branched polymer, graft copolymer, blend, mixture, and/or adduct of any of the foregoing and other polymers.

Those skilled in the art will recognize that the polymers listed herein represent an exemplary, not comprehensive, list of polymers that can be of use in accordance with the present invention.

Non-Polymeric Particles

In some embodiments, particles can be non-polymeric particles (e.g., metal particles, quantum dots, ceramic particles, polymers comprising inorganic materials, bone-derived materials, bone substitutes, viral particles, liposomes, etc.). In some embodiments, a therapeutic, diagnostic, and/or prophylactic agent to be delivered can be associated with the surface of such a non-polymeric particle. In some embodiments, a non-polymeric particle is an aggregate of non-polymeric components, such as an aggregate of metal atoms (e.g., gold atoms). In some embodiments, a therapeutic, diagnostic, and/or prophylactic agent to be delivered can be associated with the surface of, encapsulated within, surrounded by, and/or dispersed throughout an aggregate of non-polymeric components.

In some embodiments, non-polymeric particles comprise liposomes. In some embodiments, liposomes comprise a bilayer membrane. In some embodiments, liposomes comprise phospholipids (e.g., naturally-derived, synthetically-derived), optionally with mixed lipid chains (e.g., egg phosphatidylethanolamine) In some embodiments, liposomes comprise surfactant components (e.g., dioleoylphosphatidylethanolamine) In some embodiments, liposomes comprise an aqueous core. In some embodiments, micelles are lipid spheres that contain no aqueous core and are of use in accordance with the invention. In some embodiments, reverse micelles which comprise an aqueous core can be of use in accordance with the present invention. In some embodiments, liposomes can be used to deliver an agent by diffusion rather than by direct cell fusion. In some embodiments, liposomes can be used to deliver an agent to a cellular target by fusion of the lipid bilayer with other bilayers (e.g., cell membrane), thus delivering liposome contents to the cell. In some embodiments, liposomes that contain low or high pH environments can be constructed such that dissolved aqueous agents to be delivered will be charged in solution (i.e., the pH is outside the agent's pI range). As pH naturally neutralizes within the liposome (protons can pass through some membranes), the agent is neutralized, allowing it to freely pass through a membrane.

In certain embodiments of the invention, non-polymeric particles comprise gradient or homogeneous alloys. In certain embodiments of the invention, particles are composite particles made of two or more materials, of which one, more than one, or all of the materials possess an optically or magnetically detectable property, as discussed in further detail below.

In certain embodiments of the invention, particles comprise silica (SiO₂). For example, a particle may consist at least in part of silica, e.g., it may consist essentially of silica or may have an optional coating layer composed of a different material. In some embodiments, a particle has a silica core and an outside layer composed of one or more other materials. In some embodiments, a particle has an outer layer of silica and a core composed of one or more other materials. The amount of silica in the particle, or in a core or coating layer comprising silica, can range from approximately 5% to approximately 100% by mass, volume, or number of atoms, or can assume any value or range between approximately 5% and approximately 100%.

Preparation of Particles

Particles (e.g., nanoparticles, microparticles, etc.) may be prepared using any method known in the art. For example, particulate formulations can be formed by methods as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanoparticles have been described (Pellegrino et al., 2005, Small, 1:48; Murray et al., 2000, Ann. Rev. Mat. Sci., 30:545; and Trindade et al., 2001, Chem. Mat., 13:3843).

In certain embodiments, particles are prepared by the nanoprecipitation process or spray drying. Conditions used in preparing particles may be altered to yield particles of a desired size or property (e.g., hydrophobicity, hydrophilicity, external morphology, “stickiness,” shape, etc.). The method of preparing the particle and the conditions (e.g., solvent, temperature, concentration, air flow rate, etc.) used may depend on the therapeutic or diagnostic agent to be delivered and/or the composition of the polymer matrix.

Methods for making microparticles for delivery of encapsulated agents are described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755).

If particles prepared by any of the above methods have a size range outside of the desired range, particles can be sized, for example, using a sieve.

Surfactants

In some embodiments, particles may optionally comprise one or more surfactants. In some embodiments, a surfactant can promote the production of particles with increased stability, improved uniformity, or increased viscosity. Surfactants can be particularly useful in embodiments that utilize two or more dispersion media. The percent of surfactant in particles can range from 0% to 99% by weight, from 10% to 99% by weight, from 25% to 99% by weight, from 50% to 99% by weight, or from 75% to 99% by weight. In some embodiments, the percent of surfactant in particles can range from 0% to 75% by weight, from 0% to 50% by weight, from 0% to 25% by weight, or from 0% to 10% by weight. In some embodiments, the percent of surfactant in particles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

Any surfactant known in the art is suitable for use in making particles in accordance with the present invention. Such surfactants include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span 85) glycocholate; sorbitan monolaurate (Span 20); polysorbate 20 (Tween-20); polysorbate 60 (Tween-60); polysorbate 65 (Tween-65); polysorbate 80 (Tween-80); polysorbate 85 (Tween-85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof The surfactant component may be a mixture of different surfactants. These surfactants may be extracted and purified from a natural source or may be prepared synthetically in a laboratory. In certain specific embodiments, surfactants are commercially available.

Those skilled in the art will recognize that this is an exemplary, not comprehensive, list of substances with surfactant activity. Any surfactant may be used in the production of particles to be used in accordance with the present invention.

Lipids

In some embodiments, particles may optionally comprise one or more lipids. The percent of lipid in particles can range from 0% to 99% by weight, from 10% to 99% by weight, from 25% to 99% by weight, from 50% to 99% by weight, or from 75% to 99% by weight. In some embodiments, the percent of lipid in particles can range from 0% to 75% by weight, from 0% to 50% by weight, from 0% to 25% by weight, or from 0% to 10% by weight. In some embodiments, the percent of lipid in particles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

In some embodiments, lipids are oils. In general, any oil known in the art can be included in particles. In some embodiments, an oil may comprise one or more fatty acid groups or salts thereof In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof In some embodiments, a fatty acid group may be a C₁₅-C₂₀ fatty acid or salt thereof In some embodiments, a fatty acid group may be a C₁₅-C₂₅ fatty acid or salt thereof In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

In some embodiments, the oil is a liquid triglyceride.

Suitable oils for use with the present invention include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils, and combinations thereof. Suitable oils for use with the present invention include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In some embodiments, a lipid is a hormone (e.g., estrogen, testosterone), steroid (e.g., cholesterol, bile acid), vitamin (e.g., vitamin E), phospholipid (e.g., phosphatidyl choline), sphingolipid (e.g., ceramides), or lipoprotein (e.g., apolipoprotein).

Carbohydrates

In some embodiments, particles may optionally comprise one or more carbohydrates. The percent of carbohydrate in particles can range from 0% to 99% by weight, from 10% to 99% by weight, from 25% to 99% by weight, from 50% to 99% by weight, or from 75% to 99% by weight. In some embodiments, the percent of carbohydrate in particles can range from 0% to 75% by weight, from 0% to 50% by weight, from 0% to 25% by weight, or from 0% to 10% by weight. In some embodiments, the percent of carbohydrate in particles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

Carbohydrates may be natural or synthetic. A carbohydrate may be a derivatized natural carbohydrate. In certain embodiments, a carbohydrate is a monosaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a disaccharide, including but not limited to lactose, sucrose, maltose, trehalose, and cellobiose. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In certain embodiments, the carbohydrate is a sugar alcohol, including but not limited to mannitol, sorbitol, xylitol, erythritol, maltitol, and lactitol.

### Agents To Be Delivered

Agents to be incorporated in the controlled release polymer system and delivered to a target cell and/or tissue by a drug delivery system of the present invention may be therapeutic, diagnostic, prophylactic, and/or prognostic agents. Any chemical compound to be administered to an individual may be delivered using the inventive system. An agent may be a small molecule, organometallic compound, radionuclide, nucleic acid, protein, peptide, polynucleotide, carbohydrate, lipid, metal, isotopically labeled chemical compound, drug, vaccine, immunological agent, contrast agent, etc. In certain embodiments, an agent to be delivered is a small molecule (e.g., a drug). In certain embodiments, the drug is an anti-atherosclerotic agent (e.g., beta-blockers, cholesterol lowering agents, etc.). In some embodiments, the drug is a cholesterol lowering agent (e.g., lovastatin, pravastatin, simvastatin, fluvastatin, atorvastatin, niacin, etc.). In some embodiments, the drug is an anti-inflammatory agent (e.g., prednisone; dexamethasone, fluorometholone; prednisolone; methylprednisolone; clobetasol; halobetasol; hydrocortisone; triamcinolone; betamethasone; fluocinolone; fluocinonide; loteprednol; medrysone; rimexolone; celecoxib; folic acid; diclofenac; diflunisal; fenoprofen; flurbiprofen; indomethacin; ketoprofen; meclofenamate; meclofamate; piroxicam; sulindac; salsalate; nabumetone; oxaprozin; tolmetin; hydroxychloroquine sulfate; rofecoxib; etanercept; infliximab; leflunomide; naproxen; oxaprozin; piroxicam; salicylates; valdecoxib; sulfasalazine; methylprednisolone; ibuprofen; budesonide, meloxicam; methylprednisolone acetate; gold sodium thiomalate; aspirin; azathioprine; triamcinolone acetonide; propxyphene napsylate/apap; folate; nabumetone; diclofenac; ketorolac; piroxicam; etodolac; diclofenac sodium; diclofenac potassium; oxaprozin; methotrexate; minocycline; tacrolimus (FK-506); sirolimus (rapamycin) and rapamycin analogs; phenylbutazone; diclofenac sodium/misoprostol; acetaminophen; indomethacin; glucosamine sulfate/chondroitin; cyclosporin, etc.). In some embodiments, the drug is an anti-platelet agent (e.g., aspirin, clopidogrel, ticlopidine, dipyridamole, glycoprotein IIb/IIIa receptor blocker [e.g., abciximab, eptifibatide, tirofiban], cilostazol, etc.). In some embodiments, the drug is an anti-coagulant (e.g., warfarin, acenocoumarol, phenprocoumon, phenindione, heparin, low molecular weight heparin, fondaparinux, etc.). In some embodiments, the drug is an anti-proliferative agent (e.g., alkylating agents, antimetabolites, plant alkaloids, vinca alkaloids, taxanes, podophyllotoxin, topoisomerase inhibitors, hormonal therapy, antitumor antibiotics, etc.). In some embodiments, the drug is a cytotoxic agent. In certain embodiments, the drug is an immunosuppressant (e.g., glucocorticoids, cytostatics [e.g., alkylating agents, methotrexate, azathioprine, mercaptopurine], antibodies, cyclosporin, tacrolimus, sirolimus, interferons, opiods, TNF binding proteins, mycophenolate, etc.). In certain embodiments, the agent is a drug approved by the United States Food and Drug Administration (U.S.F.D.A.) for human or veterinary use. In certain embodiments, the agent is a peptide or protein. In certain embodiments, the agent is an immunogenic peptide or protein. In some embodiments, the agent is DNA. In some embodiments, the agent is RNA. In certain embodiments, the agent is an RNAi-inducing entity (e.g., siRNA, shRNA, miRNA, etc.).

In certain embodiments, the agent to be delivered is an agent designed to prevent the restenosis of a blood vessel such as a coronary artery. Examples of restenosis agents include anti-angiogenic agents such as anti-invasive factors (Eisentein et al., 1975, Am. J. Pathol., 81:337; Langer et al., 1976, Science, 193:70; Horton et al., 1978, Science, 199:1342; all of which are incorporated herein by reference); retinoic acid and derivatives thereof which alter the metabolism of extracellular matrix components to inhibit angiogenesis; tissue inhibitor of metalloproteinase-1; tissue inhibitor of metalloproteinase-2; plasminogen activator inhibitor-1; plasminogen activator inhibitor-2; and anginex (Griffioen et al., 2001, Biochem. J., 354:233; incorporated herein by reference); collagen inhibitors such as halofuginone or batimistat; antisense oligonucleotides directed to nucleic acid sequences encoding c-myc or c-myb; growth factor inhibitors such as tranilast, trapidil, or angiopeptin; antioxidants such as probucol; anti-thromobotics such as heparin or abciximab; anti-proliferative agents such as AG-1295 (Fishbein et al., 2000, Arterioscler. Thromb. Vasc. Biol., 20:667; incorporated herein by reference), tyrphostin (Banai et al., 2005, Biomaterials, 26:451; incorporated herein by reference); pacitaxel or other taxanes (Scheller et al., 2004, Circulation, 110:810; incorporated herein by reference), isoflavones (Kanellakis et al., 2004, Atherosclerosis, 176:63; incorporated herein by reference); rapamycin or derivatives or analogs thereof (Schachner et al., 2004, Ann. Thorac. Surg., 77:1580; incorporated herein by reference); vincristine; vinblastine; HMG-CoA reductase inhibitors; doxorubicin; colchicines; actinomycin D; mitomycin C; cyclosporine; or mycophenolic acid; and anti-inflammatory agents such as dexamethasone (Liu et al., 2004, Expert Rev. Cardiovasc. Ther., 2:653; incorporated herein by reference), methylprednisolone, or γ interferon; and the like which exhibit antirestenotic activity.

In some embodiments, therapeutic agents that can be utilized in accordance with the present invention include anti-proliferative, anti-neoplastic, and/or chemotherapeutic agents to prevent or treat tumors. Representative examples of such agents include androgen inhibitors; antiestrogens and hormones (e.g., flutamide, leuprolide, tamoxifen, estradiol, estramustine, megestrol, diethylstilbestrol, testolactone, goserelin, medroxyprogesterone); cytotoxic agents (e.g., altretamine, bleomycin, busulfan, carboplatin, carmustine[BiCNU], cisplantin, cladribine, dacarbazine, dactinomycin, daunorubicin, doxorubicin, estramustine, etoposide, lomustine, cyclophosphamide, cytarabine, hydroxyurea, idarubicin, interferon α-2a and -2b, ifosfamide, mitoxantrone, mitomycin, paclitaxel, streptozocin, teniposide, thiotepa, vinblastine, vincristine, vinorelbine, etc.); antimetabolites and antimitotic agents (e.g., floxuridine, 5-fluorouracil, fluarabine, interferon α-2a and -2b, leucovorin, mercaptopurine, methotrexate, mitotane, plicamycin, thioguanine, colchicines, etc.); folate antagonists and other anti-metabolites; vinca alkaloids; nitrosoureas; DNA alkylating agents; purine antagonists and analogs; pyrimidine antagonists and analogs; alkyl solfonates; enzymes (e.g., asparaginase, pegaspargase, etc.); and toxins (e.g., ricin, abrin, diphtheria toxin, cholera toxin, botulinum toxin, gelonin, pokeweed antiviral protein, tritin, Shigella toxin, and Pseudomonas exotoxin A, etc.).

In some embodiments, therapeutic agents that can be utilized within the present invention include cardiovascular agents such as antihypertensive agents; adrenergic blockers and stimulators (e.g., doxazosin, guanadrel, guanethidine, pheoxybenzamine, terazosin, clonidine, guanabenz, etc.); α- and/or β-adrenergic blockers (e.g., labetalol, etc.); angiotensin converting enzyme (ACE) inhibitors (e.g., benazepril, catopril, lisinopril, ramipril, etc.); ACE-receptor antagonists (e.g., losartan, etc.); beta blockers (e.g., acebutolol, atenolol, carteolol, pindolol, propranolol, penbatolol, nadolol, etc.); calcium channel blockers (e.g., amiloride, bepridil, nifedipine, verapamil, nimodipine, etc.); antiarrythmics, groups I-IV (e.g., bretylium, lidocaine, mexiletine, quinidine, propranolol, verapamil, diltiazem, trichlormethiazide, metoprolol tartrate, carteolol hydrochloride, etc.); and/or miscellaneous antiarrythmics and cardiotonics (e.g., adenosine, digoxin, caffeine, dopamine hydrochloride, digitalis, etc.). In some embodiments, a therapeutic agent in accordance with the present invention is not a cytokine. In some embodiments, a therapeutic agent in accordance with the present invention is not erythropoietin. In some embodiments, a therapeutic agent in accordance with the present invention is not a protein.

In some embodiments, therapeutic agents that can be used in accord with the present invention include anti-inflammatory agents. Representative examples of such agents include nonsteroidal agents (NSAIDS) such as salicylates, diclofenac, diflunisal, flurbiprofen, ibuprofen, indomethacin, mefenamic acid, nabumetone, naproxen, piroxicam, ketoprofen, ketorolac, sulindac, tolmetin, etc.

In some embodiments, anti-inflammatory drugs include steroidal agents such as beclomethasone, betamethasone, cortisone, dexamethasone, fluocinolone, flunisolide, hydorcortisone, prednisolone, prednisone, etc Immunosuppressive agents are contemplated (e.g., adenocorticosteroids, cyclosporin, etc.).

In some embodiments, therapeutic agents include anti-tissue damage agents. Representative examples of such agents include superoxide dismutase; immune modulators (e.g., lymphokines, monokines, interferons α and β, etc.); and growth regulators (e.g., IL-2, tumor necrosis factor, epithelial growth factor, somatrem, fibronectin, GM-CSF, CSF, platelet-derived growth factor, somatotropin, rG-CSF, epidermal growth factor, IGF-1, etc.).

In some embodiments, the therapeutic agent is an anti-restenotic agent such as rapamycin (i.e., sirolimus, etc.) or a derivative or analog thereof, e.g., everolimus, tacrolimus, etc. (Grube et al., 2004, Circulation, 109:2168; and Grube and Buellesfeld, 2004, Herz, 29:162; both of which are incorporated herein by reference). In some embodiments, the therapeutic agent is an anti-apoptotic agent such as Galectin-3; (−)deprenyl; monoamine oxidase inhibitors (MAO-I) such as selegiline and rasagiline; rapamycin; querceten, etc.

By way of example, the following classes of drugs or the drug in a polymeric drug delivery system may be conjugated to FcRn binding partners for the purposes of delivery to epithelial and/or endothelial cells:

Antineoplastic Compounds. nitrosoureas, e.g., carmustine, lomustine, semustine, strepzotocin; methylhydrazines, e.g., procarbazine, dacarbazine; steroid hormones, e.g., glucocorticoids, estrogens, progestins, androgens, tetrahydrodesoxycaricosterone, cytokines and growth factors; asparaginase;

Immunoactive Compounds. Immunosuppressives, e.g., pyrimethamine, trimethopterin, penicillamine, cyclosporine, azathioprine; immunostimulants, e.g., levamisole, diethyl dithiocarbamate, enkephalins, endorphins;

Antimicrobial Compounds. Antibiotics, e.g., β lactam, penicillin, cephalosporins, carbapenims and monobactams, β-lactamase inhibitors, aminoglycosides, macrolides, tetracyclins, spectinomycin; Antimalarials, Amebicides, Antiprotazoal, Antifungals, e.g., amphotericin B, Antiviral, e.g., acyclovir, idoxuridine, ribavirin, trifluridine, vidarbine, gancyclovir;

Parasiticides. Antihalmintics, Radiopharmaceutics, gastrointestinal drugs. Hematologic Compounds. Immunoglobulins; blood clotting proteins; e.g., antihemophilic factor, factor IX complex; anticoagulants, e.g., dicumarol, heparin Na; fibrolysin inhibitors, tranexamic acid;

Cardiovascular Drugs. Peripheral antiadrenergic drugs, centrally acting antihypertensive drugs, e.g., methyldopa, methyldopa HCl; antihypertensive direct vasodilators, e.g., diazoxide, hydralazine HCl; drugs affecting renin-angiotensin system; peripheral vasodilators, phentolamine; antianginal drugs; cardiac glycosides; inodilators; e.g., amrinone, milrinone, enoximone, fenoximone, imazodan, sulmazole; antidysrhythmic calcium entry blockers; drugs affecting blood lipids; ranitidine, bosentan, rezulin;

Respiratory Drugs. Sypathomimetic drugs: albuterol, bitolterol mesylate, dobutamine HCl, dopamine HCl, ephedrine So, epinephrine, fenfluramine HCl, isoproterenol HCl, methoxamine HCl, norepinephrine bitartrate, phenylephrine HCl, ritodrine HCl; cholinomimetic drugs, e.g., acetylcholine Cl; anticholinesterases, e.g., edrophonium Cl; cholinesterase reactivators; adrenergic blocking drugs, e.g., acebutolol HCl, atenolol, esmolol HCl, labetalol HCl, metoprolol, nadolol, phentolamine mesylate, propanolol HCl; antimuscarinic drugs, e.g., anisotropine methylbromide, atropine SO₄, clinidium Br, glycopyrrolate, ipratropium Br, scopolamine HBr;

Neuromuscular Blocking Drugs. Depolarizing, e.g., atracurium besylate, hexafluorenium Br, metocurine iodide, succinylcholine Cl, tubocurarine Cl, vecuronium Br; centrally acting muscle relaxants, e.g., baclofen;

Neurotransmitters and neurotransmitter agents;

Acetylcholine, adenosine, adenosine triphosphate, amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K⁺ channel toxins;

Antiparkinson Drugs. amaltidine HCl, benztropine mesylate, e.g., carbidopa;

Diuretic Drugs. Dichlorphenamide, methazolamide, bendroflumethiazide, polythiazide;

Uterine, Antimigraine Drugs. Carboprost tromethamine, mesylate, methysergide maleate;

Hormones. Pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists, hormonal contraceptives, testicular hormones; gastrointestinal hormones: cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, sincalide;

Enzymes. Hyaluronidase, streptokinase, tissue plasminogen activator, urokinase, PGE-adenosine deaminase;

Intravenous Anesthetics. Droperidol, etomidate, fetanyl citrate/droperidol, hexobarbital, ketamine HCl, methohexital Na, thiamylal Na, thiopental Na;

Antiepileptics. Carbamazepine, clonazepam, divalproex Na, ethosuximide, mephenytoin, paramethadione, phenytoin, primidone;

Peptides and proteins. ankyrins, arrestins, bacterial membrane proteins, clathrin, connexins, dystrophin, endothelin receptor, spectrin, selectin, cytokines; chemokines; growth factors, insulin, erythropoietin (EPO), tumor necrosis factor (TNF), neuropeptides, neuropeptide Y, neurotensin, transforming growth factor α, transforming growth factor β, interferon (IFN), and hormones, growth inhibitors, e.g., genistein, steroids etc.; glycoproteins, e.g., ABC transporters, platelet glycoproteins, GPIb-IX complex, GPIIb-IIIa complex, vitronectin, thrombomodulin, CD4, CD55, CD58, CD59, CD44, lymphocye function-associated antigen, intercellular adhesion molecule, vascular cell adhesion molecule, Thy-1, antiporters, CA-15-3 antigen, fibronectins, laminin, myelin-associated glycoprotein, GAP, GAP-43. In certain embodiment of the present invention, the polypeptide therapeutics may be covalently conjugated to the FcRn binding partner or the FcRn binding partner and therapeutic may be expressed as a fusion protein using standard recombinant genetic techniques;

Cytokines and Cytokine Receptors. Examples of cytokines and receptors thereof which may be delivered via a FcRn binding partner or conjugated to an FcRn binding partner in accordance with the present invention, include, but are not limited to: Interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor, macrophage colony stimulating factor, platelet derived growth factor, stem cell factor, tumor growth factor β, tumor necrosis factor, lymphotoxin, Fas, granulocyte colony stimulating factor, granulocyte macrophage colony stimulating factor, interferon α, interferon β, interferon γ;

Growth Factors and Protein Hormones. Examples of growth factors and receptors thereof and protein hormones and receptors thereof which may be delivered via a FcRn binding partner or conjugated to an FcRn binding partner in accordance with the present invention, include, but are not limited to: erythropoietin, angiogenin, hepatocyte growth factor, fibroblast growth factor, keratinocyte growth factor, nerve growth factor, tumor growth factor a, thrombopoietin, thyroid stimulating factor, thyroid releasing hormone, neurotrophin, epidermal growth factor, VEGF, ciliary neurotrophic factor, LDL, somatomedin, insulin growth factor, insulin-like growth factor I and II;

Chemokines. Examples of chemokines and receptors thereof which may be delivered via a FcRn binding partner or conjugated to an FcRn binding partner in accordance with the present invention, include, but are not limited to: ENA-78, ELC, GRO-α, GRO-β, GRO-γ, HRG, LIF, IP-10, MCP-1, MCP-2, MCP-3, MCP-4, MIP-1α, MIP-1β, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2, α-chemokine receptors: CXCR1, CXCR2, CXCR3, CXCR4, CXCR5, CXCR6, CXCR7 β-chemokine receptors: CCR1, CCR2, CCR3, CCR4, CCR5, CCR6, CCR7;

Chemotherapeutics. The FcRn binding partners may be conjugated to chemotherapy or anti-tumor agents which are effective against various types of human cancers, including leukemia, lymphomas, carcinomas, sarcomas, myelomas etc., such as, doxorubicin, paclitaxel, mitomycin, cisplatin, daunorubicin, bleomycin, actinomycin D, neocarzinostatin;

Antibodies. The FcRn binding partners of the present invention may be conjugated to antibodies including, but not limited to: (a) anti-cluster of differentiation antigen CD-1 through CD-166 and the ligands or counter receptors for these molecules; (b) anti-cytokine antibodies, e.g., anti-IL-1 through anti-IL-18 and the receptors for these molecules; (c) anti-immune receptor antibodies, antibodies against T cell receptors, major histocompatibility complexes I and II, B cell receptors, selectin killer inhibitory receptors, killer activating receptors, OX-40, MadCAM-1, Gly-CAM1, integrins, cadherens, sialoadherens, Fas, CTLA-4, Fcy-receptors, Fcα-receptors, Fcε-receptors, Fcμ-receptors, and their ligands; (d) anti-metalloproteinase antibodies, e.g., collagenase, MMP-1 through MMP-8, TIMP-1, TIMP-2; anti-cell lysis and/or proinflammatory molecules, e.g., perforin, complement components, prostanoids, nitron oxide, thromboxanes; and (e) anti-adhesion molecules, e.g., carcioembryonic antigens, lamins, fibronectins; and

Antiviral Agents. The FcRn binding partners may be conjugated to antiviral agents such as reverse transcriptase inhibitors and nucleoside analogs, e.g., ddI, ddC, 3TC, ddA, AZT; protease inhibitors, e.g., Invirase, ABT-538; inhibitors of in RNA processing, e.g., ribavirin.

Specific examples of known therapeutics which may be delivered via an FcRn binding partner include, but are not limited to: Capoten, Monopril, Pravachol, Avapro, Plavix, Cefzil, Duricef/Ultracef, Azactam, Videx, Zerit, Maxipime, VePesid, Paraplatin, Platinol, Taxol, UFT, Buspar, Serzone, Stadol NS, Estrace, Glucophage; Ceclor, Lorabid, Dynabac, Prozac, Darvon, Permax, Zyprexa, Humalog, Axid, Gemzar, Evista (Eli Lily); Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin; Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor; Vantin, Rescriptor, Vistide, Genotropin, Micronase/Glyburide/Glynase, Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine; Lopid, Accrupil, Dilantin, Cognex, Neurontin, Loestrin, Dilzem, Fempatch, Estrostep, Rezulin, Lipitor, Omnicef, FemHRT, Suramin, and Clinafloxacin.

In some embodiments, examples of therapeutic agents which may be delivered by FcRn binding partners are found in Goodman and Gilman's The Pharmacological Basis of Therapeutics, 9th ed., McGraw-Hill, 1996 (incorporated herein by reference).

In some embodiments, FcRn binding partners may be conjugated to one or more diagnostic agents, such as a pharmaceutically acceptable positron-emitting moiety; β-emitting moiety; γ-emitting moiety, including but not limited to, indium and technetium; magnetic particle; radiopaque material such as barium; and/or fluorescent compound.

### Production of Polymeric Drug Delivery Conjugates

In some embodiments, the invention provides methods of preparing inventive drug delivery systems. In some embodiments, drug delivery systems comprise a particle associated with an FcRn binding partner (e.g., Fc fragment) and a therapeutic, diagnostic, and/or prophylactic agent to be delivered. In some embodiments, drug delivery systems comprise an FcRn binding partner (e.g., Fc fragment) conjugated to a therapeutic, diagnostic, and/or prophylactic agent to be delivered.

In accordance with the present invention, any physicochemical association or attachment may be used. One of ordinary skill in the art will readily appreciate that the nature of the association will depend, among other things, upon the mode of administration and the pharmaceutical carriers used to deliver the conjugate to the selected epithelial barrier. For example, some associations or bonds are not as well suited as others to withstand certain environments such as the stomach, but can be protected by delivery systems which bypass the stomach. It is, of course, important that the bond between the FcRn binding partner and the polymeric system be of such a nature that it does not destroy the ability of the FcRn binding partner to bind to the FcRn receptor. Such bonds are well known to those of ordinary skill in the art; examples are described herein.

Inventive drug delivery systems may be manufactured using any available method. When associating FcRn binding partners (e.g., Fc fragments) and/or agents to be delivered with particles, it is desirable to have a particle which can be efficiently linked to a FcRn binding partner (e.g., Fc fragment) and/or agent to be delivered using simple chemistry without adversely affecting the function of the FcRn binding partners (e.g., Fc fragments) and/or agents to be delivered. It is desirable that the drug delivery conjugate should be able to avoid uptake by the mononuclear phagocytic system after systemic administration so that it is able to reach specific organs, tissues, and/or cells in the body.

In some embodiments, FcRn binding partners and agents to be delivered are covalently associated with particles (e.g., via amide, ester, carbon-carbon bond, disulfide bond, or other “click” chemistry). In certain embodiments, the surface of the particle is activated for covalent attachment of an FcRn binding partner and/or agent to be delivered. For example, in certain embodiments, activated electrophiles are formed on one component and reacted with nucleophiles on the other component of the system. Attaching FcRn binding partners or agents to be delivered to the particles using this technique may lead to up to 1%, up to 2%, up to 3%, up to 4%, up to 5%, or up to 10% of electrophiles or nucleophiles unreacted. For example, activated esters may be formed on the surface of the particle for attaching amines such as primary amines of lysine residues. In certain embodiments in which the agent to be delivered is a protein or peptide, the conjugate may be formed as a fusion protein. For covalently-associated drug delivery systems, release and delivery of the therapeutic, diagnostic, and/or prophylactic agent to a target site occurs by disrupting the covalent association(s). For example, if an agent is associated with a particle by a cleavable linker, the agent is released and delivered to the target site upon cleavage of the linker.

In some embodiments, a controlled release polymer system, FcRn binding partner (e.g., Fc fragment), and/or agent to be delivered are directly associated with one another, e.g., by one or more covalent bonds. In some embodiments, a controlled release polymer system, FcRn binding partner (e.g., Fc fragment), and/or agent to be delivered are associated by means of one or more linkers. In some embodiments, one or more linkers form one or more covalent or non-covalent bonds with the FcRn binding partner (e.g., Fc fragment) and the controlled release polymer system, thereby attaching them to one another. In some embodiments, one or more linkers form one or more covalent or non-covalent bonds with the agent to be delivered and the controlled release polymer system, thereby attaching them to one another.

Any suitable linker can be used in accordance with the present invention. Linkers may be used to form amide linkages, ester linkages, disulfide linkages, etc. Linkers may contain carbon atoms or heteroatoms (e.g., nitrogen, oxygen, sulfur, etc.). Typically, linkers are 1 to 50 atoms long, 1 to 40 atoms long, 1 to 25 atoms long, 1 to 20 atoms long, 1 to 15 atoms long, 1 to 10 atoms long, or 1 to 10 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In some embodiments, a linker is an aliphatic or heteroaliphatic linker. In some embodiments, the linker is a polyalkyl linker. In certain embodiments, the linker is a polyether linker. In certain embodiments, the linker is a polyethylene linker. In certain specific embodiments, the linker is a polyethylene glycol (PEG) linker.

In some embodiments, a linker is a short peptide chain, e.g., between 1 and 10 amino acids in length, e.g., 1, 2, 3, 4, or 5 amino acids in length, a nucleic acid, an alkyl chain, etc.

In some embodiments, the linker is a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g., esterase cleavable linker), ultrasound-sensitive linkers, x-ray cleavable linkers, etc. In some embodiments, the linker is not a cleavable linker.

Any of a variety of methods can be used to associate a linker with a particle or an agent to be delivered. General strategies include passive adsorption (e.g., via electrostatic interactions), multivalent chelation, high affinity non-covalent binding between members of a specific binding pair, covalent bond formation, etc. (Gao et al., 2005, Curr. Op. Biotechnol., 16:63). In some embodiments, click chemistry can be used to associate a linker with a particle (e.g., Diels-Alder reaction, Huigsen 1,3-dipolar cycloaddition, nucleophilic substitution, carbonyl chemistry, epoxidation, dihydroxylation, etc.).

A bifunctional cross-linking reagent can be employed. Such reagents contain two reactive groups, thereby providing a means of covalently associating two target groups. The reactive groups in a chemical cross-linking reagent typically belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), N-Succinimidyl 3-[2-pyridyldithio]-propionamido (SPDP), succimidyl α-methylbutanoate, biotinamidohexanoyl-6-amino-hexanoic acid N-hydroxy-succinimide ester (SMCC), succinimidyl-[(N-maleimidopropionamido)-dodecaethyleneglycol]ester (NHS-PEO12), etc. For example, carbodiimide-mediated amide formation and active ester maleimide-mediated amine and sulfhydryl coupling are widely used approaches.

Common schemes for forming a polymeric drug delivery conjugate involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g., a reagent containing both a succinimidyl ester and either a maleimide, a pyridyldisulfide, or an iodoacetamide Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to particles via amine or thiol groups in lysine or cysteine side chains respectively, or by an N-terminal amino group. Nucleic acids such as RNAs can be synthesized with a terminal amino group. A variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) may be used to associate the various components of targeted particles. Particles can be prepared with functional groups, e.g., amine or carboxyl groups, available at the surface to facilitate association with a biomolecule. Any biomolecule can be attached to a particle and/or inventive complex using any of the methods described herein.

For additional general information on association and/or conjugation methods and cross-linkers, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong S S, Chemistry of Protein Conjugation and Cross-linking, CRC Press Publishers, Boca Raton, 1991; and Hermanson, G. T., Bioconjugate Techniques, Academic Press, Inc., San Diego, 1996.

In some embodiments, FcRn binding partners and agents to be delivered are non-covalently associated with particles. For example, particles may comprise polymers, and FcRn binding partners and agents to be delivered may be associated with the surface of, encapsulated within, surrounded by, and/or distributed throughout the polymermic matrix of a particle. Examples of associations useful in the present invention include hydrophobic interactions between the delivery system and the hydrophobic portion of an antibody molecule, antibody-controlled drug delivery particle specific binding, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc. For example, particles may comprise polymers, and therapeutic, diagnostic, and/or prophylactic agents may be associated with the surface of, encapsulated within, and/or distributed throughout the polymeric matrix of a particle. Agents are released by diffusion, degradation of the particle, and/or combination thereof. In some embodiments, polymers degrade by bulk erosion. In some embodiments, polymers degrade by surface erosion.

In some embodiments, a particle may be associated with an FcRn binding partner (e.g., Fc fragment) and/or agent to be delivered via electrostatic interactions. For example, a particle may have a cationic surface or may be reacted with a cationic polymer, such as poly(lysine) or poly(ethylene imine), to provide a cationic surface. The particle surface can then bind via electrostatic interactions with a negatively charged FcRn binding partner (e.g., Fc fragment). For example, a portion of the FcRn binding partner (e.g., Fc fragment) may be attached to a negatively charged polymer (e.g., a poly(carboxylic acid)) that can interact with the cationic polymer surface without disrupting the binding affinity of the FcRn binding partner (e.g., Fc fragment) for its target.

In some embodiments, a particle may be associated with an FcRn binding partner (e.g., Fc fragment) and/or agent to be delivered via affinity interactions. For example, biotin may be attached to the surface of the particle and streptavidin may be attached to the FcRn binding partner (e.g., Fc fragment); or conversely, biotin may be attached to the FcRn binding partner (e.g., Fc fragment) and the streptavidin may be attached to the surface of the particle. The biotin group and streptavidin are typically attached to the particle, FcRn binding partner, or agent to be delivered via a linker, such as an alkylene linker or a polyether linker. Biotin and streptavidin bind via affinity interactions, thereby binding the particle to the complex. Other specific binding pairs could be similarly used (e.g., histidine-tagged biomolecules can be associated with particles conjugated to nickel-nitrolotriaceteic acid (Ni-NTA)).

In some embodiments, a particle may be associated with an FcRn binding partner (e.g., Fc fragment) and/or agent to be delivered via metal coordination. For example, a polyhistidine may be attached to one end of FcRn binding partner (e.g., Fc fragment), and a nitrilotriacetic acid can be attached to the surface of the particle. A metal, such as Ni²⁺, chelates the polyhistidine and the nitrilotriacetic acid, thereby binding the FcRn binding partner (e.g., Fc fragment) to the particle.

In some embodiments, a particle may be associated with an FcRn binding partner (e.g., Fc fragment) and/or agent to be delivered via physical adsorption. For example, an FcRn binding partner (e.g., Fc fragment) may comprise a hydrophobic tail, such as polymethacrylate or an alkyl group having at least about 10 carbons. The hydrophobic tail typically adsorbs onto the surface of a hydrophobic particle, such as a particle comprising a polyorthoester, polysebacic anhydride, or polycaprolactone, thereby binding the FcRn binding partner (e.g., Fc fragment) to the particle.

In some embodiments, a particle may be associated with an FcRn binding partner (e.g., Fc fragment) and/or agent to be delivered via host-guest interactions. For example, a macrocyclic host, such as cucurbituril or cyclodextrin, may be attached to the surface of the particle and a guest group, such as an alkyl group, a polyethylene glycol, or a diaminoalkyl group, may be attached to the FcRn binding partner (e.g., Fc fragment); or conversely, the host group may be attached to the FcRn binding partner (e.g., Fc fragment) and the guest group may be attached to the surface of the particle. In some embodiments, the host and/or the guest molecule may be attached to the FcRn binding partner (e.g., Fc fragment) or the particle or agent via a linker, such as an alkylene linker or a polyether linker.

In some embodiments, a particle may be associated with an FcRn binding partner (e.g., Fc fragment) and/or agent to be delivered via hydrogen bonding interactions. For example, an oligonucleotide having a particular sequence may be attached to the surface of the particle, and an essentially complementary sequence may be attached to one or both ends of the FcRn binding partner (e.g., Fc fragment) such that it does not disrupt the binding affinity of the FcRn binding partner (e.g., Fc fragment) for its target. The FcRn binding partner (e.g., Fc fragment) then binds to the particle via complementary base pairing with the oligonucleotide attached to the particle. Two oligonucleotides are essentially complimentary if about 80% of the nucleic acid bases on one oligonucleotide form hydrogen bonds via an oligonucleotide base pairing system, such as Watson-Crick base pairing, reverse Watson-Crick base pairing, Hoogsten base pairing, etc., with a base on the second oligonucleotide. Typically, it is desirable for an oligonucleotide sequence attached to the particle to form at least about 6 complementary base pairs with a complementary oligonucleotide attached to the FcRn binding partner (e.g., Fc fragment).

It is to be understood that the compositions of the invention can be made in any suitable manner, and the invention is in no way limited to compositions that can be produced using the methods described herein. Selection of an appropriate method may require attention to the properties of the particular moieties being associated.

If desired, various methods may be used to separate controlled release polymer systems having at least one attached FcRn binding partner (e.g., Fc fragment) and/or agent to be delivered from controlled release polymer systems to which an FcRn binding partner (e.g., Fc fragment) and/or agent to be delivered has not become attached, or to separate controlled release polymer systems having different numbers of FcRn binding partners (e.g., Fc fragments) and/or agents to be delivered attached thereto. For example, size exclusion chromatography, agarose gel electrophoresis, or filtration can be used to separate populations of controlled release polymer systems having different numbers of FcRn binding partners (e.g., Fc fragments) and/or agents to be delivered attached thereto and/or to separate controlled release polymer systems from other entities. Some methods include size-exclusion or anion-exchange chromatography.

### Pharmaceutical Compositions

The inventive drug delivery system may be administered per se (neat) or in the form of a pharmaceutically acceptable salt, solution, suspension, or solid form. When used in medicine, the composition should be pharmaceutically acceptable. Pharmaceutically acceptable salts include, but are not limited to, those prepared from the following acids: hydrochloric, hydrobromic, sulphuric, nitric, phosphoric, maleic, acetic, salicyclic, p-toluene sulphonic, tartaric, citric, methane sulphonic, formic, acetate, malonic, succinic, naphthalene-2-sulphonic, fatty acid (e.g., palmitate), and benzene sulphonic. In some embodiments, pharmaceutically acceptable salts can be prepared as alkaline metal or alkaline earth salts, such as sodium, potassium, magnesium, or calcium salts of the carboxylic acid group.

Components of the pharmaceutical compositions are capable of being comingled with the drug delivery system of the present invention, and with each other, in a manner such that there is no interaction which would substantially impair the desired pharmaceutical efficiency. Components of oral drug formulations include diluents, binders, lubricants, glidants, disintegrants, coloring agents, and flavoring agents. Encapsulating substances for the preparation of enteric-coated oral formulations include cellulose acetate phthalate, polyvinyl acetate phthalate, hydroxypropyl methylcellulose phthalate, and methacrylic acid ester copolymers. Solid oral formulations such as capsules or tablets are sometimes preferred. Elixirs and syrups are well known oral formulations. Components of aerosol formulations include solubilized active ingredients, antioxidants, solvent blends, and propellants for solution formulations, and micronized and suspended active ingredients, dispersing agents and propellants for suspension formulations. Oral, aerosol and nasal formulations of the invention can be distinguished from injectable preparations of the prior art because such formulations may be nonaseptic, whereas injectable preparations are typically aseptic.

The pharmaceutical compositions of this invention can be administered to a patient by any means known in the art including orally, inhalationally (e.g., in an aerosol), locally (stent, catheters, inflated balloon delivery devices, etc.), intravascularly, systemically, and parenterally. The term “subject,” as used herein, refers to humans as well as non-humans, including, for example, mammals, birds, reptiles, amphibians, and fish. Preferably, non-humans are mammals (e.g., a rodent, a mouse, a rat, a rabbit, a monkey, a dog, a cat, a primate, or a pig). In certain embodiments parenteral routes are preferred since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), by inhalation (as by sprays) or locally by any means such self-inflated balloon and/or catheters. Pharmaceutical compositions for oral administration can be liquid or solid. The methods of this invention, generally speaking, involve delivering the drug delivery system of the invention to an epithelial and/or endothelial surface. In certain embodiments, the drug delivery system is delivered to the endothelial surface of the cardiovasculature using a catheter. In certain embodiments, a balloon catheter is used. In some embodiments, preferred modes of administration of biologically active substances are oral, intrapulmonary, intrabiliary, intravenously, locally, and intranasal. For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone, methylcellulose, or hydroxypropyl methylcellulose); fillers (e.g., lactose, microcrystalline cellulose, or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, hydrogenated edible fats, etc.); emulsifying agents (e.g., lecithin, acacia, etc.); non-aqueous vehicles (e.g., almond oil, oily esters, ethyl alcohol, fractionated vegetable oils, etc.); and/or preservatives (e.g., methyl or propyl-p-hydroxybenzoates, sorbic acid, etc.). Preparations may contain buffer salts, flavoring, coloring and sweetening agents as appropriate. Preparations for oral, local, and/or systemic administration may be suitably formulated to give controlled release of the active ingredient. By way of example, but not by limitation, FcRn binding partners may be conjugated to the following therapeutics for epithelial and/or endothelial barrier targeted delivery.

For administration by inhalation, an active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

A device for the local delivery of a drug delivery formulation into a natural tissue conduit in the mammalian body, having a first element capable of contacting the lumen of the conduit and a second element which overlays first element, a reservoir being formed between the first element and the second element, the interior of the reservoir being capable of fluid communication with the conduit such that a substance placed in the reservoir is delivered into the conduit. The present invention provides methods of locally delivering a pharmaceutical composition into a natural tissue conduit in the mammalian body using the drug delivery system of the present invention. Drug delivery combinations and associated local delivery devices provide a means for overcoming the difficulties associated with the methods and devices currently in use. In addition, methods for maintaining the drug delivery formulation on the local delivery device ensure that the drug delivery formulation combinations reach the target site.

In accordance with some embodiments, the present invention provides a local drug delivery apparatus. In some embodiments, a local drug delivery apparatus comprises a medical device for implantation into a treatment site of a living organism and at least one agent in therapeutic dosages releasable affixed to the medical device for the treatment of reactions by the living organism caused by the medical device or the implantation thereof. In some embodiments, a local delivery apparatus comprises a material for preventing the at least one agent from separating from the medical device prior to implantation of the medical device at the treatment site, the material being affixed to at least one of the medical device or a delivery system for the medical device.

In some embodiments, a local drug delivery apparatus comprises a medical device for implantation into a treatment site of a subject and at least one agent in therapeutic dosages releasably affixed to the medical device, the at least one agent being incorporated into a polymeric matrix. In some embodiments, a local drug delivery apparatus optionally comprises a material for preventing the at least one agent from separating from the medical device prior to implantation of the medical device at the treatment site, the material being affixed to at least one of the medical device or a delivery system for the medical device.

Drug delivery formulations may be affixed to any number of medical devices to treat various diseases. A drug delivery formulation may be affixed to minimize or substantially eliminate the biological organism's reaction to the introduction of the medical device utilized to treat a separate condition. For example, stents, catheters, and/or balloons, self-expandable or nor, degradable or not, may be introduced to open coronary arteries or other body lumens such as biliary ducts. The introduction of these stents causes a smooth muscle cell proliferation effect as well as inflammation. Accordingly, the stents, catheters, and/or balloons may be coated with drug delivery formulations to combat these reactions.

In some embodiments, drug delivery formulations should preferably remain on the medical devices during delivery and implantation. Accordingly, various coating techniques for creating strong bonds between the drug delivery may be utilized. Alternatively or additionally, various materials may be utilized as surface modifications to prevent the drug delivery formulation from coming off prematurely. Drug delivery formulation using self-inflated balloons or other medical devices could be used to create a pressure released system to local tissues.

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn oil, germ oil, olive oil, castor oil, sesame oil, etc.), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds of the invention are mixed with solubilizing agents such as Cremophor, alcohols, oils, modified oils, glycols, polysorbates, cyclodextrins, polymers, and combinations thereof. In certain embodiments, the compound is mixed with an alcohol, such as ethanol, and Cremophor (polyethoxylated castor oil).

Injectable preparations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils may be employed as a solvent or suspending medium. For this purpose any bland fixed oil can be employed including synthetic mono- or diglycerides. In addition, fatty acids such as oleic acid may be used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively or additionally, delayed absorption of a parenterally administered drug form is accomplished by dissolving or suspending the drug in an oil vehicle. Depot injectable formulations may be prepared by entrapping the drug in liposomes or microemulsions which are compatible with body tissues.

Compositions for rectal or vaginal administration are usually suppositories which can be prepared by mixing the compounds of this invention with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally contain opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

Active ingredients can be in micro-encapsulated form with one or more excipients as noted above. Solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally contain opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are contemplated as being within the scope of this invention. In some embodiments, the present invention contemplates the use of transdermal patches, which have the added advantage of providing controlled delivery of a compound to the body. Such dosage forms can be made by dissolving or dispensing the compound in the proper medium. Absorption enhancers can be used to increase the flux of the compound across the skin. The rate can be controlled by either providing a rate controlling membrane or by dispersing the compound in a polymer matrix or gel.

### Administration

The invention provides methods of using inventive drug delivery systems. An inventive drug delivery system may be delivered via any known route. In certain embodiments, it is delivered orally. In some embodiments, it is delivered inhalationally. In some embodiments, it is delivered parenterally. In some embodiments, it is delivered intravascularly. The system is particularly useful for administering agents across a cell layer. In particular, the system is useful for delivering agents across the endothelial cell layer of the vasculature. An effective amount of a controlled drug delivery system conjugated to an FcRn binding partner is administered to an animal in need of such treatment. In certain embodiments, the system is delivered directly to an endothelial or epithelial cell layer. In certain embodiments, the system is delivered via a catheter (e.g., a balloon catheter).

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like. In certain embodiments of the invention, inventive drug delivery systems as described herein are formulated by conjugating with water soluble chelators, or water soluble polymers such as polyethylene glycol as poly (1-glutamic acid), or poly (1-aspartic acid), as described in U.S. Pat. No. 5,977,163 (incorporated herein by reference).

In certain embodiments, drug delivery systems of the invention may be administered orally or parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of subject body weight per day, one or more times a day, to obtain the desired therapeutic effect. The desired dosage may delivered as delivered every other day, every third day, every week, every two weeks, every three weeks, or every four weeks. In certain embodiments, the desired dosage may be delivered using multiple administrations (e.g., two, three, four, five, six, seven, eight, nine, or ten administrations).

In some embodiments, the drug delivery system and/or pharmaceutical compositions of the present invention can be employed in combination therapies, that is, the system and pharmaceutical compositions can be administered concurrently with, prior to, or subsequent to, one or more other desired therapeutic agents and/or medical procedures. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In general, each agent will be administered at a dose and/or on a time schedule determined for that agent. In some embodiments, the invention encompasses the delivery of inventive conjugates and/or pharmaceutical compositions in combination with agents that may improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same disorder, or they may achieve different effects (e.g., control of any adverse effects). In some embodiments, compositions of the invention are administered with a second therapeutic agent that is approved by the U.S. Food and Drug Administration.

In some embodiments, therapeutically active agents utilized in combination may be administered together in a single composition. In some embodiments, therapeutically active agents utilized in combination may be administered separately in different compositions. In some embodiments, the present invention encompasses “therapeutic cocktails” comprising inventive compositions.

In general, it is expected that agents utilized in combination with be utilized at levels that do not exceed the levels at which they are utilized individually. In some embodiments, the levels utilized in combination will be lower than those utilized individually.

Conjugates and/or pharmaceutical compositions of the present invention may be administered alone and/or in combination with any other therapeutic, diagnostic, and/or prophylactic agents (for example, any of the therapeutic agents described herein).

One of ordinary skill in the art will understand that the examples presented above are not meant to be limiting. The principles presented in the examples above can be generally applied to any combination therapies for treatment of any disease, disorder, and/or condition.

### Kits

In some embodiments, the present invention provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use, or sale of pharmaceutical products, which notice reflects approval by the agency of manufacture, use or sale for human administration. For example, the invention provides kits comprising inventive conjugates and/or pharmaceutical compositions and instructions for use. A kit may comprise multiple different drug delivery systems. A kit may comprise any of a number of additional components or reagents in any combination. All of the various combinations are not set forth explicitly but each combination is included in the scope of the invention.

According to certain embodiments of the invention, a kit may include, for example, (i) a drug delivery system; (ii) instructions for administering the system to a subject in need thereof.

In certain embodiments, a kit may include, for example, (i) at least one agent to be delivered; (ii) at least one FcRn binding partner (e.g., Fc fragment); (iii) a polymeric matrix precursor; and (iv) instructions for assembling inventive drug delivery systems from individual components (i)-(iii).

Kits typically include instructions for use of inventive drug delivery systems (e.g. particles, conjugates) and/or pharmaceutical compositions. Instructions may, for example, comprise protocols and/or describe conditions for production of drug delivery systems, administration of systems to a subject in need thereof, etc. Kits generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

These and other aspects of the present invention will be further appreciated upon consideration of the following Examples, which are intended to illustrate certain particular embodiments of the invention but are not intended to limit its scope, as defined by the claims.

### Examples

### Example 1

### FcRn Targeted Nanoparticles

The synthesis of a multi-block polymer is initiated by conjugation of functionalized biodegradable polyesters with chemical groups such as, but not limited to, maleimide or carboxylic acid for easy conjugation to one end of thiol, amine, or similarly functionalized polyethers. Conjugation of polymer to the antibody fragment is performed in aqueous buffer including phosphate buffers, Tris buffers, etc. The other free end of the polyether is functionalized with chemical groups for conjugation to a library of targeting moieties such as antibodies and/or derivatives thereof. An antibody may be conjugated through a functional group including but not limited to thiol, amine, carboxylates, hydroxyls, aldehydes, ketones, and photoreactions. The conjugation reaction between a targeting moieties and the poly-ester-ether copolymer is achieved by adding antibody molecules dissolved in aqueous solution. Biodegradable and biocompatible polymer poly(lactide-co-glycolide) (PLGA)/PLA and polyethylene glycol (PEG) can be used as a model for the block copolymer of poly(ester-ether). In a representative embodiment, the FcRn receptor can be used for dynamic transport targeting using an Fc fragment as the targeting moieties to cancer cells. Carboxylic acid modified PLGA (PLGA-COOH) or PLA is conjugated to the amine modified heterobifunctional PEG and form a copolymer of PLGA-PEG. By using a modified Fc fragment, a polymer of PLGA/PLA-PEG-Fc fragment conjugate is obtained by conjugating the end of PEG and the modified Fc fragment. Any crosslinking agent may be used provided that a) the activity of the compound is retained, and b) binding by the FcRn of the Fc portion of the conjugate is not adversely affected. The polymer conjugate can be useful for imaging and diagnostic applications. In such embodiments, a photo-sensitive or environmental-responsive compound will be linked to the multiblock polymer.

Targeted nanoparticles are formed by precipitation of the multi-block polymer in an aqueous environment and subsequently conjugated to the polymeric nanoparticles. The nanoparticle formulation system described here is compatible with high throughput biological assays in order to test the nanoparticles generated from the multi-block polymer. It is possible to control the density on the surface and to optimize the formulation polymer/affibody for therapeutic application.

### Materials

Poly(D,L-lactide)-block-poly(ethylene glycol) was synthesized by ring opening polymerization. Carboxylic acid terminal poly(D,L-lactide) and/or poly(lactic-co-glycolic acid) was purchased from the DURECT corporation (Pelham, Ala.). PEG was purchased from Nektar Therapeutics (San Carlos, Calif.). Fc fragment was purchased from (Sweden). All other reagents were purchased from Sigma Aldrich.

### Triblock Polymer Synthesis

Poly(ethylene glycol)-block-poly(D,L-lactic acid), COOH-poly(ethylene glycol)-block-poly(D,L-lactic acid) (COOH-PEG-PLA), and methoxypoly(ethylene glycol)-block-poly(D,L-lactic acid) (mPEG-PLA) were synthesized by ring opening polymerization in anhydrous toluene using tin(II) 2-ethylhexanoate as catalyst. General procedure for syntheses of the copolymers is as follows. D,L-Lactide (1.6 g, 11.1 mmol) and PEG₃₅₀₀ (0.085 mmol) in anhydrous toluene (10 mL) was heated to reflux temperature (about 120° C.), after which polymerization was initiated by adding tin(II) 2-ethylhexanoate (20 mg). After stirring for 9 hours with reflux, the reaction mixture was cooled to room temperature. Cold water (10 mL) was added to this solution and the resulting suspension was stirred vigorously at room temperature for 30 minutes to hydrolyze unreacted lactide monomers. The resulting mixture was transferred to separate funnel containing CHCl₃ (50 mL) and water (30 mL). After layer separation, organic layer was collected, dried using anhydrous MgSO₄, filtered, and concentrated under reduced vacuum. Then, hexane was added to the concentrated solution to precipitate polymer product. Pure PEG₃₅₀₀-PLA or PEG₃₅₀₀-PLGA was collected as a white solid. Both copolymers were characterized by ¹H-NMR (400 MHz, Bruker Advance DPX 400) and gel permeation chromatography (GPC) (Waters Co, Milford, Mass., USA).

Alternatively, the conjugation of PLGA or PLA and PEG were achieved in the presence of 1-ethyl-3-[3-dimethylaminopropyl]carbodiimide hydrochloride) (EDC) and N-hydroxysuccinimide (NHS). Briefly, PLGA particles were dissolved in acetonitrile. The carboxylic end of PLGA was activated by mixing with NHS and EDC at a molar ratio of COOH to EDC and NHS and stir overnight at room temperature. Excess EDC and NHS in the solution was quenched by adding 2-mercaptoethanol. NHS-activated PLGA was purified by precipitation in a solution containing ethyl ether and methanol, and followed by centrifugation at 3000×g for 10 minutes. To conjugate the amine end of NH₂-PEGs with the NHS-activated PLGA, both polymers were mixed at a molar ratio of 1:1.3 (PLGA-NHS:NH₂-PEGs) at room temperature overnight. The resulting PLGA-PEG copolymer was purified by precipitation in ethyl ether-methanol solution. The conjugation of the end of PEG-PLGA and the modified Fc fragment was performed.

Nanoparticles were formed by precipitating the copolymer in water. Briefly, the polymer was dissolved in acetonitrile, and then mixed slowly with water. Nanoparticles formed instantly upon mixing. The residual acetonitrile in the suspension was evaporated by continuously stirring the suspension at room temperature for 4 hours. Subsequently, nanoparticles are functionalized with Fc fragment in aqueous solution.

### Example 2

### Transcytosis of Drug Delivery Systems

### Materials and Methods

Materials

All chemicals and reagents were obtained from the following commercial sources: purified human IgG-Fc, Bethyl Laboratories Inc. (Montgomery, Tex.); D,L-lactide monomer, Boehringer Ingelheim (Ingelheim, Germany); bifunctional polyethylene glycol (PEG) with terminal hydroxyl and maleimide functional groups (OH-PEG3400-MAL), Nektar Therapeutics (Santa Carlos, Calif.); anhydrous toluene, Sigma-Aldrich (St Louis, Mo.); tin(II) 2-ethylhexanoate Sigma-Aldrich (St Louis, Mo.); Amicon Ultra devices with 100 kD molecular weight size exclusion, Millipore (Carrigtwohill, Ireland).

Polymer Synthesis

PLA-PEG-MAL was synthesized by ring opening polymerization in anhydrous toluene using tin(II) 2-ethylhexanoate as a catalyst. Briefly, D,L-Lactide (1.6 g, 11.1 mmol) and MAL-PEG₃₅₀₀-OH (0.085 mmol) in anhydrous toluene (10 ml) was heated to reflux temperature (˜120 ° C.), after which the polymerization was initiated by adding tin(II) 2-ethylhexanoate (20 mg). After stirring with reflux, the reaction mixture was cooled to room temperature. Cold water (10 ml) was added to this solution was and the resulting suspension was stirred vigorously at room temperature for 30 minutes to hydrolyze unreacted lactide monomers. The mixture was transferred to a separate funnel containing CHCl₃ (50 ml) and water (30 ml). After phase separation, the organic layer was collected, dried using anhydrous MgSO₄, filtered, and concentrated under reduced vacuum. Pure polymer was collected as a white solid and characterized by ¹H-NMR for characteristic peak chemical shift values of PLA (400 MHz, Bruker Advance DPX 400), δ=5.28-5.11 (m; —OC—CH(CH₃)—O—; PLA) and PEG, δ=3.62 (s; —CH₂—CH₂—O—; PEG).

Preparation of Nanoparticles

The nanoprecipitation method was employed for the formation of nanoparticle formulations. Briefly, PLA-PEG-MAL (10 kD) was dissolved in acetonitrile (5 mg/ml) and ³H-PLGA. Nanoparticles (NP) were formed by adding the polymer mixture solution to water, a non-solvent. The resulting radiolabeled PLA-PEG-MAL NP suspension was allowed to stir uncovered for 6 hours at room temperature. NP formulations were purified by centrifugation using Amicon Ultra centrifuge devices (100 kD molecular weight size exclusion). The NP formulations were re-suspended, washed with water, and collected using the same centrifugation method.

Preparation of Nanoparticle-Fc Bioconjugates

PLA-PEG-MAL nanoparticles prepared as described in the section above were incubated under stirring conditions with the Traut's reagent derivatized IgG-Fc molecule (40 kD) at a molar ratio of PLA-PEG-MAL/Fc of 5%-10% to form a stable bioconjugate. Subsequently, nanoparticle-Fc bioconjugates were purified from free Fc molecules using Amicon Ultra centrifuge devices (100 kD molecular weight size exclusion) prior to further studies.

In Vitro Transcytosis Assays of Targeted and Non-Targeted Nanoparticles

Transepithelial electrical resistance (TEER) measurements using Millicell-ERS (Millipore) were performed on Transwell® plates with a cell density of 5.5×104 cells (HUVEC and Caco-2) in their respective growth media. The medium was replaced with Hank's Balanced Salt Solution (HBSS) at pH 6.5 on the apical side and pH 7.4 on the basolateral side of the transwells and let to equilibrate for 1 hour at 37° C. The integrity of the monolayers was measured by TEER prior to the transport experiment. The apical solutions were then replaced either by targeted nanoparticles or its respective control (non-targeted) in a solution of HBSS at pH 6.5. The concentration of nanoparticles in solution was 400 μg/ml. Nanoparticle suspensions were incubated for 3 hours. Samples from the basolateral side were then collected, dissolved in scintillation solvent, and vortexed. ³H was detected (DPM count) using a scintillation counter. After the experiment, the integrity of the cell monolayer was again measured using TEER.

In Vivo Transcytosis Assays of Targeted and Non-Targeted Nanoparticles

Balb/c mice (n=3/group) were fasted overnight prior to gavage with nanoparticle formulations. The nanoparticles (5 mg) were prepared as described above and suspended in 250 μl of protease inhibitor solution composed of casein and aprotinin in distilled water. Gavage was performed using an animal feeding stainless steel. One hour after gavage, duodenal tissue was collected, washed, fixed with para-formaldehyde (4%), frozen into block and cryosectioned for microscope imaging. Nanoparticle formulations were visualized by fluorescent microscopy (DeltaVision system). Digital imaging of light field and red (Cy5 fluorescent dye) fluorescence were acquired along their z-axis at 0.2 μm intervals under oil immersion at 10× magnification.

Results

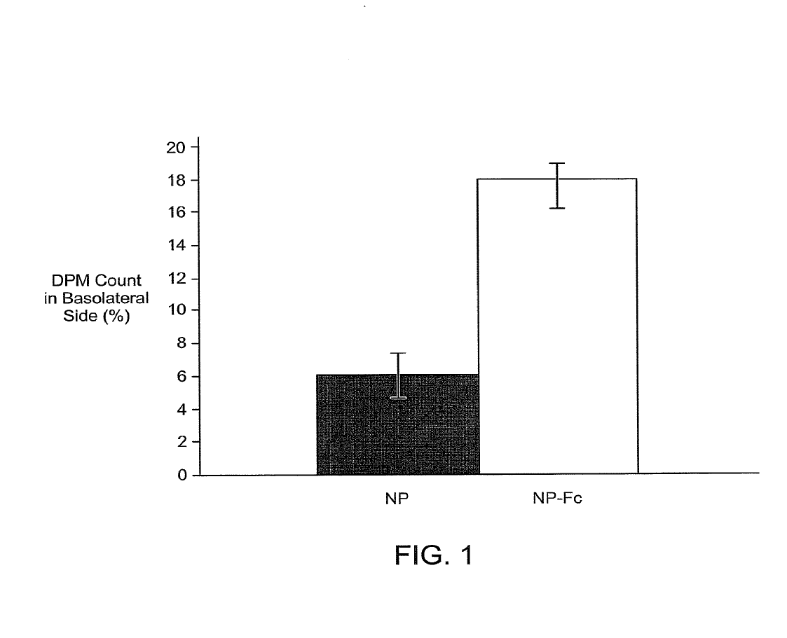

A monolayer of human umbelical vein cells (HUVEC) were grown on transwell forming tight junctions (TEER=˜170 Ω/cm²). Targeted (NP-Fc) and non-targeted (NP) nanoparticles were incubated on the apical side of transwells and collected from the basolateral side. FIG. 1 shows the percentage of nanoparticles that was collected from the basolateral side. Targeted nanoparticles are transported more efficiently (˜18%) than non-targeted nanoparticles (˜6%).

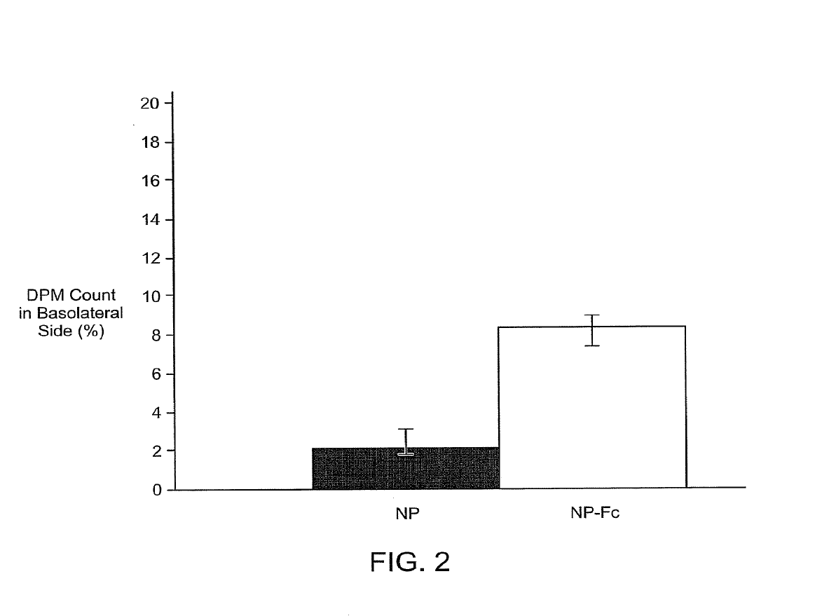

A monolayer of epithelial cells (Caco-2) were grown on transwell forming tight junctions (TEER=˜1000 Ω/cm²). Targeted (NP-Fc) and non-targeted (NP) nanoparticles were incubated on the apical side of transwells and collected from the basolateral side. FIG. 2 shows the percentage of nanoparticles that was collected from the basolateral side. Targeted nanoparticles are transported more efficiently (˜10%) than non-targeted nanoparticles (˜2%).

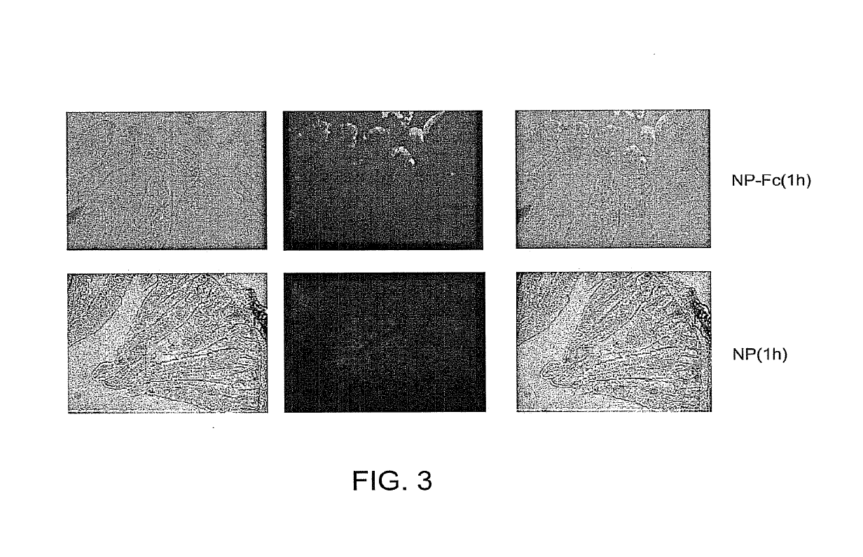

Nanoparticles were fluorescently labeled for imaging. Five milligrams of nanoparticles with or without Fc in solution with protease inhibitors (250 ul) was gavaged into Balb/C mice (n=1). Duodenal tissues were collected 1 hour after gavage, fixed with para-formaldehyde, frozen into block and cryosectioned prior to fluorescent imaging. The fluorescent images represent the uptake of targeted and non-targeted fluorescent nanoparticles (red) 1 hour after gavage. FIG. 3 shows that nanoparticles with Fc are targeting the intestine.

### Example 3

### Multifunctional, Responsive Nanoparticles for Oral Protein Delivery

In general, some major obstacles for oral protein delivery include enzymatic protein degradation and poor intestinal epithelium permeability. Polymeric nanoparticle delivery systems with the IgG Fc fragment conjugated to the surface could potentially overcome both barriers. Proteins encapsulated within nanoparticles would be shielded from the acidic environment and digestive enzymes present in the gastrointestinal tract. Interactions of FcRn binding partners (e.g., Fc fragments) with the FcRn provide a potential mechanism for crossing the epithelial barrier using the transcytosis route.

The present invention encompasses the recognition that the use of IgG Fc fragment as a targeting moiety offers a potential method to overcome the epithelium permeability issue. However, use of an IgG Fc fragment may enhance the immune system barrier present in the gastrointestinal tract. Receptors for IgG Fc are expressed by many different types of immune cells present in the lamina propria, including antigen-presenting cells such as dendritic cells and macrophages. The FcRn itself is expressed on macrophages and some subsets of dendritic cells. Nanoparticles with Fc molecules on the surface would appear similar to an antibody-covered virus and could be endocytosed while diffusing through the lamina propria. Nanoparticles able to enter the bloodstream would have to pass by macrophages in the liver while nanoparticles that enter the lacteal would have to pass by macrophages in the lymph nodes. The present invention encompasses the recognition that nanoparticles may shed the Fc fragment from the surface before encountering immune cells in order to minimize the risk of uptake by immune cells.

Based on this criterion, the present invention encompasses the recognition that a nanoparticle delivery system design may comprise a nanoparticle with IgG Fc conjugated to the surface for transport across the intestinal epithelium. After transcytosis, the Fc fragment may optionally be shed based on some stimuli (e.g., pH or enzymatic cleavage), thereby exposing a different surface functionality such as polyethylene glycol (PEG) that could minimize interactions with immune cells. The change in surface functionality may happen before the nanoparticles enter the lamina propria and encounter immune cells. However, nanoparticles typically remain attached to the Fc-FcRn complex in order to be correctly trafficked across the cell. In specific embodiments, the Fc fragment is not shed until exocytosis from the cell.

The present invention encompasses the recognition that one possible approach is the use of a second targeting moiety on the nanoparticle surface that allows adhesion to a component of the extracellular matrix (ECM) in the basal lamina. Adhesion to a component of the basal lamina may prevent nanoparticles from entering the lamina propria, minimizing encounters with immune cells. Uptake by cells may be more difficult because of steric hindrances due to nanoparticle anchoring to the ECM. After anchoring, nanoparticles may release the cargo into the extracellular environment, which could then diffuse to and directly enter the bloodstream. To further reduce the possibility of encounters with immune cells, in some embodiments, an Fc fragment could be cleaved from the nanoparticle surface while anchored to the basal lamina using enzymes present in the extracellular environment.

The basal lamina is a continuous sheet of ECM that underlies the basolateral surface of epithelial cells. In the intestinal epithelium, the basal lamina comprises laminins, collagens (predominately type IV), proteoglycans, fibulin, and other proteins. While any of these components could potentially be targeted, collagen IV represents ˜50% of all proteins in the basal lamina The present invention encompasses the recognition that collagen IV may be a good target for the second targeting moiety to anchor nanoparticles to the basal lamina One potential candidate for the second targeting moiety is the CREKA peptide, which binds to non-helical collagen such as collagen IV. It has the added advantages of being linear, only 5 amino acids long, and easy to conjugate since the sulfhydryl group of the cysteine is not required for binding activity. Proteins such as integrins could also be used for this purpose.

The components of the ECM are continually degraded and reconstructed. This is accomplished by a family of zinc-containing enzymes called matrix metalloproteinases (MMPs) that are either secreted into the extracellular space or bound to the external surface of the plasma membrane. MMP-2 (gelatinase A) and MMP-9 (gelatinase B) are secreted MMPs responsible for the degradation of collagen IV. The present invention encompasses the recognition that these enzymes could potentially be used to cleave the Fc fragment from the nanoparticle surface. Work with peptide libraries has identified 8-mer peptides that are cleaved by MMP-2 with higher kinetic rates than collagen and could be used as an enzyme-cleavable linker for the Fc fragment (Turk et al., 2001, Nat. Biotechnol., 19:661; incorporated herein by reference).

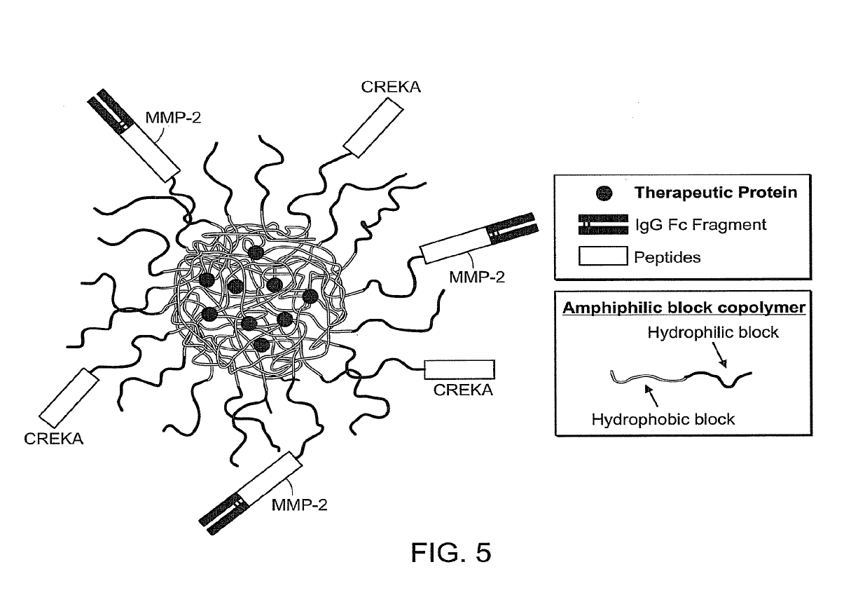

The present invention encompasses the recognition that one potential design for the delivery system based on the components discussed is a nanoparticle with two different surface functionalities (FIG. 5). The nanoparticle may be formed from PLGA-PEG or PLA-PEG copolymers through self-assembly. The first surface functionality may be the IgG Fc fragment linked to the surface by a peptide that is susceptible to cleavage by MMP-2. The second functionality may be the CREKA peptide. The IgG Fc may allow the nanoparticles to cross the intestinal epithelium through the transcytosis route. Once across, the CREKA peptide may allow the nanoparticles to anchor to collagen IV in the basal lamina MMPs present in the ECM may then degrade the Fc linker, shedding the Fc from the nanoparticle surface and reducing the probability of uptake by immune cells. Finally, the protein cargo may be released from the nanoparticles and diffuse to the capillary beds for entry into the bloodstream.

Polymer Synthesis

The polymer used can either be poly(D,L-lactic acid) (PLA) or poly(D,L-lactic-co-glycolic acid) (PLGA). Poly(ethylene glycol) (PEG) can be conjugated to PLA or PLGA to create diblock copolymers. The length of the PEG block can be used to control the distance between different ligands and the end groups can have several different functionalities, providing flexibility in the conjugation chemistries possible for ligand attachment.

Poly(D,L-lactic acid)-block-polyethylene glycol-COOH (PLA-PEG-COOH) can be synthesized using D,L-lactide and OH-PEG-COOH by ring opening polymerization. PLGA-PEG-COOH can be synthesized using PLGA-COOH and NH₂-PEG-COOH by conjugation with EDC/NHS chemistry. Diblock copolymers can be characterized by NMR for chemical structure and gel permeation chromatography (GPC) for molecular weight distribution.

Nanoparticle Surface Functionalization

Any available approach and/or chemistry may be used for the development of multifunctional nanoparticles. In some embodiments, the initial approach used is to conjugate the two targeting moieties (CREKA peptide and MMP-2 peptide-Fc) to separate batches of nanoparticles, followed by the mixing of these batches to form multifunctional nanoparticles. This approach can simplify the ligand conjugation chemistries and allow for characterization of each ligand separately before combination.

Many different analytical tools can be used to characterize the functionalized nanoparticles. Dynamic light scattering measures particle size and surface charge. NMR is used to determine the chemical structure. Ligand conjugation reactions can be measured using protein or peptide kits depending on the ligand. Alternatively, fluorescent dyes attached to the ligands can be used to monitor the conjugation.

Activity assays can be used to determine ligand activity after conjugation to nanoparticles. For the CREKA peptide, a collagen-binding assay can be developed. Collagen IV-coated microplates together with fluorescently-labeled CREKA or dye-encapsulated CREKA nanoparticles are used for binding quantification and imaging of nanoparticles on the collagen surface. For the IgG Fc ligand, a binding assay can also be performed. One possibility is the use of human IgG Fc ELISA microplates. As with the CREKA peptide, fluorescently-labeled IgG Fc ligand or dye-encapsulated nanoparticles with Fc can be used for binding quantification. A MMP-2 enzyme assay can be performed for the MMP-2 cleavable peptide. The MMP-2 enzyme is commercially available and can be used for kinetic studies. Enzyme is added to peptide solutions to determine the peptide cleavage kinetics prior to conjugation. Cleavage reactions are monitored by following the production of amine using fluorescamine. After conjugation, kinetics are monitored by attaching a fluorescent dye or fluorescently-labeled Fc to the peptide. This assay can be used in combination with the binding assays to measure cleavage kinetics while the nanoparticles are attached to a surface.

For CREKA peptide conjugation, the first step may be nanoparticle formation using the nanoparecipitation method. In this method, diblock copolymer is dissolved in an organic solvent. This solution is then added to an aqueous phase, causing the self-assembly of nanoparticles due to the amphiphilic nature of the copolymer with PEG extending into the aqueous phase and exposing a functional end group. The size of the nanoparticles can be controlled using parameters such as the organic solvent, polymer concentration, and ratio of organic to aqueous phases to form nanoparticles less than 100 nm in diameter. For the CREKA peptide conjugation, the PEG end group may be maleimide, which reacts with thiol groups to form stable carbon-sulfur bonds. The cysteine residue on the CREKA peptide has a thiol group that is not required for binding activity and will be used to conjugate CREKA to the nanoparticle. After conjugation, the surface coverage of the peptide is quantified based on the conjugation reaction efficiency calculated using a peptide quantification kit. With the mass of peptide conjugated to the surface and polymer mass along with the respective molecular weights, the peptide surface coverage can be estimated. The nanoparticle-bound CREKA peptide is then tested for activity using the collagen-binding assay. Nanoparticles with and without CREKA peptide on the surface are compared using the assay to determine the binding enhancement due to the CREKA peptide conjugation.

In some embodiments, the MMP-2 cleavage peptide is attached to nanoparticles using the same conjugation method used for the CREKA peptide. A cysteine residue may be added to the peptide for conjugation with the PEG maleimide end group assuming that the cysteine residue does not affect the peptide cleavage kinetics. Otherwise, alternate conjugation chemistries are available. Peptide conjugation is quantified using a peptide quantification kit and used to estimate the peptide surface coverage. The MMP-2 enzyme assay is then used to determine the affect of conjugation of the peptide's cleavability using a fluorescent dye attached to the peptide for quantification of the kinetics. The peptide conjugation is followed by Fc fragment conjugation to the exposed C-terminus of the peptide using EDC/NHS chemistry. The conjugation is quantified with a protein quantification kit and used to estimate the Fc surface coverage. Conjugated Fc fragment activity is measured using the Fc binding assay and compared with nanoparticles without Fc fragment. While the nanoparticles are attached to the surface, MMP-2 enzyme is added to determine the cleavability of the MMP-2 cleavage peptide. Particle size is measured after conjugation of each ligand to determine the affect on size.

After forming both types of nanoparticles and evaluating ligand activity, in some embodiments, the next step is to mix the two types together to form multifunctional nanoparticles. Multifunctional nanoparticles are formed using the nanoprecipitation method. Prior to mixing, the CREKA-nanoparticles are solubilized in an organic solvent such as dimethyl sulfoxide (DMSO). The MMP2-Fc-nanoparticles can also be solublized in DMSO. However, the effect of DMSO exposure on the activity of the Fc fragment will have to be determined If there is a significant affect on Fc activity, then the MMP2-Fc-nanoparticles may remain in water. The two types of nanoparticles are mixed together with a high volume fraction of DMSO to allow mixing of the soluble polymers before nanoprecipitation. The volume fraction of DMSO is optimized to allow mixing of the polymers while retaining Fc fragment activity in the water fraction.

In some embodiments, multifunctional nanoparticles in the mixture may be isolated using a selection process specific for both ligands. This can be achieved by using the Fc binding assay to collect all nanoparticles with active Fc fragment on the surface. Nanoparticles can then be recovered and applied to the collagen binding assay. Particles that bind in both assays have both ligands in active states. The surface coverage of Fc fragment is determined using GPC. The cleavage kinetics of the MMP-2 peptide is measured for nanoparticles in solultion or attached to a surface using the MMP-2 cleavage assay. The Fc fragment can then be cleaved from the nanoparticles and peptide quantification can be used to determine the surface coverage of the peptides.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. For example, it is to be understood that any of the compositions of the invention can be used for inhibiting the formation, progression, and/or recurrence of adhesions at any of the locations, and/or due to any of the causes discussed herein or known in the art. It is to be understood that any of the compositions made according to the methods for preparing compositions disclosed herein can be used for inhibiting the formation, progression, and/or recurrence of adhesions at any of the locations, and/or due to any of the causes discussed herein or known in the art. The present invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention (e.g., any hydrogel precursor, any polysaccharide derivative or non-polysaccharide polymer, e.g., any HA derivative or cellulose derivative, any molecular weight range, any cross-linking agent, any type of covalent bond between hydrogel precursors, any class of biologically active agent or specific agent, any particle size and/or material composition, any route or location of administration, any purpose for which a composition is administered, etc.), can be excluded from any one or more claims. For example, in certain embodiments of the invention the biologically active agent is not an anti-proliferative agent. For purposes of brevity, all of the embodiments in which one or more elements, features, purposes, or aspects is excluded are not set forth explicitly herein.

1. A particle comprising an agent encapsulated in a particle, wherein the particle is associated with an FcRn binding partner.

2. The particle of claim 1, wherein the FcRn binding partner is an IgG Fc fragment.

3-6. (canceled)

7. The particle of claim 2, wherein the IgG Fc fragment comprises an amino acid sequence that is at least 80% identical to any of SEQ ID NOs.: 1-6.

8. The particle of claim 2, wherein the IgG Fc fragment comprises at least about 20 contiguous amino acids of any of SEQ ID NOs.: 1-6.

9-13. (canceled)

14. The particle of claim 1, whereby the FcRn binding partner allows the particle to bind to FcRn receptor.

15. The particle of claim 1, whereby the FcRn binding partner allows the particle to bind to FcRn receptor of endothelial or epithelial cells.

16. The particle of claim 1, whereby the FcRn binding partner allows the particle to cross a cell layer by transcytosis.

17. The particle of claim 1, wherein the FcRn binding partner allows the particle to cross an epithelial or endothelial barrier by transcytosis.

18. The particle of claim 1, wherein the agent is a therapeutic, diagnostic, prognostic, or prophylactic agent.

19. The particle of claim 1, wherein the agent is a therapeutic agent.

20. The particle of claim 19, wherein the agent is selected from the group consisting of anti-atherosclerotic agents, cholesterol-lowering agents, thrombolytic agents, anti-platelet agents, and anti-proliferative agents.

21-25. (canceled)

26. The particle of claim 1, wherein the agent is selected from the group consisting of insulin, human growth hormone, erythropoietin, cytokines, interferons, antibodies, monoclonal antibodies, humanized antibodies, antibody fragments, protein C, thrombin, bone morphogenetic proteins, colony-stimulating factor, etanercept, and enzymes.

27. The particle of claim 1, wherein the particle is in a form suitable for oral administration or pulmonary administration.

28. (canceled)

29. The particle of claim 1, wherein the particle is a polymeric particle.

30. The particle of claim 29, wherein the polymeric particle comprises a polymer selected from the group consisting of polyalkylenes, polycarbonates, polyanhydrides, polyhydroxyacids, polyfumarates, polycaprolactones, polyamides, polyacetals, polyethers, polyesters, poly(orthoesters), polyvinyl alcohols, polyurethanes, polyphosphazenes, polyacrylates, polymethacrylates, polycyanoacrylates, polyureas, polystyrenes, polyamines, poly(arylates), polycarbonates, polypropylene fumarates), polyhydroxyalkanoates, polyketals, polyesteramides, poly(dioxanones), polyhydroxybutyrates, polyhydroxyvalyrates, polyorthocarbonates, polyvinyl pyrrolidone), polyalkylene oxalates, polyalkylene succinates, poly(malic acid), poly(methyl vinyl ether), and poly(maleic anhydride).

31. (canceled)

32. The particle of claim 29, wherein the polymer is selected from the group consisting of polylactic acid, polyglycolic acid, poly(lactic-co-glycolic acid), polyvalerolactone, poly(1,3-dioxan-2one), poly(sebacic anhydride), and polyethylene glycol.

33. (canceled)

34. The particle of claim 1, wherein the particle is a non-polymeric particle selected from the group consisting of a metal particle, a quantum dot, a ceramic particle, an inorganic particle, a bone particle, a liposome, a micelle, and a reverse micelle.

35. (canceled)

36. The particle of claim 1, wherein the FcRn binding partner is covalently attached to the particle.

37. (canceled)

38. The particle of claim 1, wherein the FcRn biding partner is covalently or non-covalently associated with the particle in a orientation-specific manner.

39. The particle of claim 1, wherein the FcRn binding partner is non-covalently associated with the particle through any association selected from the group consisting of affinity interactions, metal coordination, physical adsorption, host-guest interactions, hydrophobic interactions, pi stacking interactions, hydrogen bonding interactions, van der Waals interactions, magnetic interactions, electrostatic interactions, and dipole-dipole interactions.

40. The particle of claim 1, wherein the particle is less than 1000 nm in diameter.

41-46. (canceled)

47. A conjugate for drug delivery comprising a therapeutic, diagnostic, prognostic, or prophylactic an agent associated with an IgG Fc fragment FcRn binding partner, wherein the IgG Fc fragment comprises an amino acid sequence that is at least 80% identical to any of SEQ ID NOs.: 1-6 or comprises at least about 20 contiguous amino acids of any of SEQ ID NOs.: 1-6.

48-65. (canceled)

66. The conjugate of claim 47, wherein the agent is selected from the group consisting of anti-atherosclerotic agents, cholesterol-lowering agents, thrombolytic agents, anti-platelet agents, anti-coagulants, and anti-proliferative agents.

67-71. (canceled)

72. The conjugate of claim 47, wherein the agent is selected from the group consisting of insulin, human growth hormone, interferons, antibodies, monoclonal antibodies, humanized antibodies, antibody fragments, protein C, thrombin, bone morphogenetic proteins, colony-stimulating factor, etanercept, and enzymes.

73. The conjugate of claim 47, wherein the particle is in a form suitable for oral or pulmonary administration.

74. (canceled)

75. The conjugate of claim 47, wherein the FcRn binding partner is covalently attached to the agent.

76. The conjugate of claim 47, wherein the FcRn binding partner is non-covalently associated with the agent through any association selected from the group consisting of affinity interactions, metal coordination, physical adsorption, host-guest interactions, hydrophobic interactions, pi stacking interactions, hydrogen bonding interactions, van der Waals interactions, magnetic interactions, electrostatic interactions, and dipole-dipole interactions.

77. (canceled)

78. The conjugate of claim 47, wherein the FcRn biding partner is covalently or non-covalently associated with the agent in a orientation-specific manner.

79. The particle of claim 1, wherein the particle is associated with a binding partner of an adhesion molecule.

80. The particle of claim 79, wherein the adhesion molecule is selected from the group consisting of selectins, integrins, immunoglobulin superfamily members, and cadherins.

81. The particle of claim 79, wherein the adhesion molecule is selected from the group consisting of ICAM-I, ICAM-2, VCAM-I, E-selectin, and P-selectin.

82-85. (canceled)

86. The conjugate of claim 47, wherein the conjugate is encapsulated in a polymeric particle, wherein the particle is associated with a binding partner of an adhesion molecule.

87. The conjugate of claim 86, wherein the adhesion molecule is selected from the group consisting of selectins, integrins, immunoglobulin superfamily members, and cadherins.

88. The conjugate of claim 86, wherein the adhesion molecule is selected from the group consisting of ICAM-I, ICAM-2, VCAM-I, E-selectin, and P-selectin.

89-94. (canceled)

95. A pharmaceutical composition comprising an agent associated with an FcRn binding partner.

96-99. (canceled)

100. A method of administering a particle, the method comprising step of: administering a particle of claim 1 to a subject in need thereof.

101. The method of claim 100, wherein the step of administering comprises administering the particle orally, intravascularly, or via inhalation.

102-107. (canceled)

108. A method of preparing the particle of claim 1, the method comprising steps of: providing a particle comprising an agent to be delivered encapsulated in a polymeric matrix; providing an FcRn binding partner; and attaching the FcRn binding partner to the surface of the particle.

109. The method of claim 108, wherein the step of attaching comprises covalently attaching the FcRn binding partner to the surface of the particle.

110. A method of preparing the conjugate of claim 47, the method comprising steps of: providing an agent to be delivered; providing an FcRn binding partner; and associating the FcRn binding partner to the agent.

111. The method of claim 110, wherein the step of associating comprises covalently attaching the FcRn binding partner to the agent.
